SEQUENCE LISTING (11 sequences)

SEQ ID NO: 1 - protein, 12 aa, Artificial Sequence
  human APPL1 peptide
ASSRPNQASSEG

SEQ ID NO: 2 - protein, 15 aa, Artificial Sequence
  APPL1 peptide
SQSEESDLGEGGKKR

SEQ ID NO: 3 - protein, 14 aa, Artificial Sequence
  APPL1 peptide
VPDPDPTKFPVNRN

SEQ ID NO: 4 - protein, 15 aa, Artificial Sequence
  Sortilin-1 peptide
WVSKNFGGKWEEIHK

SEQ ID NO: 5 - protein, 15 aa, Artificial Sequence
  Sortilin-1 peptide
EKDYTIWLAHSTDPE

SEQ ID NO: 6 - protein, 15 aa, Artificial Sequence
  Syndecan-1 pepetide
EPKQANGGAYQKPTK

SEQ ID NO: 7 - protein, 15 aa, Artificial Sequence
  Syndecan-1 peptide
SHPHRDMQPGHHETS

SEQ ID NO: 8 - protein, 12 aa, Artificial Sequence
  Syndecan-1 peptide
TPRPRETTQLPT

SEQ ID NO: 9 - protein, 22 aa, Artificial Sequence
  APPL1 peptide
SRLIAASSRPNQASSEGQFVVL

SEQ ID NO: 10 - protein, 20 aa, Artificial Sequence
  Sortilin-1 peptide
ENGLWVSKNFGGKWEEIHKA

SEQ ID NO: 11 - protein, 21 aa, Artificial Sequence
  Syndecan-1 peptide
EPKQANGGAYQKPTKQEEFYA

(19) United States
(12) Patent Application Publication
(10) Pub. No.: US 2023/0375551 A1
(43) Pub. Date: Nov. 23, 2023

(54) METHODS FOR CONFIRMING DETECTION AND EVALUATING THE PROGRESSION OF A PROSTATE CANCER AND RELATED THERAPIES

(71) Applicant: Envision Sciences Pty Ltd, Adelaide (AU)

(72) Inventor: Doug BROOKS, Adelaide (AU)

(21) Appl. No.: 17/762,517

(22) PCT Filed: Sep. 3, 2020
(86) PCT No.: PCT/AU2020/050925

§ 371 Date: Mar. 22, 2022

(30) Foreign Application Priority Data
Sep. 3, 2019 (AU) 2019903248

(51) Int. Cl. G01N 33/574

(52) U.S. Cl. CPC G01N 33/57434; G01N 2800/52; G01N 2800/56

(57) ABSTRACT

The present invention relates to methods for detecting prostate cancer, methods for reliable prostate cancer grading, methods for determining the progression of prostate cancer, methods for defining advanced prostate cancer, and methods for treating prostate cancer patients based on the detection of biomarkers. In particular, the present disclosure is based on the determination that a combination of three specific endosomal biomarkers can be used to detect prostate cancer and/or determine the degree of progression of such cancer in a subject.

### FIELD

The present invention relates to methods for detecting prostate cancer, methods for reliable prostate cancer grading, methods for determining the progression of prostate cancer, methods for defining advanced prostate cancer, and methods for treating prostate cancer patients based on the detection of biomarkers.

### BACKGROUND

Prostate cancer is the most common form of cancer in males from developed countries, and the incidence of this disease is predicted to double globally by 2030. For example, in 2016 more than 19,300 Australian men were diagnosed with prostate cancer and over 3,000 patients died, making this disease one of the largest causes of cancer-related deaths in this country. Internationally over 1,000,000 prostate cancer patients are diagnosed each year and more than 300,000 die, making this a health care issue of global concern.

The prostate-specific antigen (PSA) test is currently used for prostate cancer screening, however, this assay suffers from a number of disadvantages, including a high percentage of false-positive and false-negative results. PSA also cannot distinguish between aggressive or more slow-growing cancers at the time of diagnosis, and result in unnecessary biopsies and in over-treatment. Recently there have been recommendations to abandon this procedure, particularly in older men.

The digital rectal examination is an alternative procedure to check the prostate for abnormalities, but this test is limited by the inability to assess the whole gland and to some degree the size of the tumour.

Once prostate cancer is suspected, a biopsy may be taken to confirm the diagnosis and to grade the cancer, but there are significant problems with tissue histology assessment and Gleason/Epstein grading particularly with inter-operator reliability. To date no specific aspects of histopathology has been identified that directly correlate with patient outcomes, although higher Gleason grades/Epstein scores are indicative of poor patient outcomes.

There is therefore an urgent need for more specific and/or more accurate detection methods for prostate cancer, to assist in diagnosis/prognosis and to enable the selection of the most appropriate therapeutic interventions. Early accurate detection significantly reduces mortality from prostate cancer, making improved diagnostic and prognostic methods an important objective.

Given the deficiencies associated with current techniques for the diagnosis/prognosis of prostate cancer, the ability to utilise biomarkers to assist in the detection of prostate cancer would be highly advantageous. However, the identification of clinically relevant biomarkers associated with prostate cancer remains problematic and it is likely that a combination of biomarkers may be needed to solve the current problems.

Accordingly, there remains a need to identify more reliable methods to detect prostate cancer as well as the progression of the cancer to an advanced stage, in order to better aid current and future treatment regimens.

### SUMMARY

The present disclosure is based on the determination that a combination of three specific endosomal biomarkers can be used to detect prostate cancer and/or determine the degree of progression of such cancer in a subject.

Certain embodiments of the present disclosure provide methods of detecting a prostate cancer in a subject, the method comprising detecting one or more of an altered presence, level, secretion and distribution of at least the following three endosomal biomarkers APPL1 (Adaptor protein, phosphotyrosine interacting with PH domain and leucine zipper 1, APPL1), Sortilin-1 (SORT1), and Syndecan-1 (SDC1) in a biological sample from the subject as compared to a reference.

In one embodiment, the reference is a normal or benign prostate tissue or a normal blood or plasma sample. The reference may also be a cut-off value.

In one embodiment, the method comprises detecting elevated levels of APPL1, SORT1 and SDC1.

In one embodiment, the method comprises detecting elevated levels of APPL1 and SDC1.

In one embodiment, the subject is one who has taken a PSA test.

Certain embodiments of the present disclosure provide methods of detecting a prostate cancer in a subject, the method comprising detecting elevated levels of at least the following three endosomal biomarkers APPL1, SORT1, and SDC1 in a biological sample from the subject as compared to a reference.

Certain embodiments of the present disclosure provide methods of detecting and measuring the severity of a prostate cancer in a subject, the method comprising detecting one or more of an altered presence, level, secretion and distribution of at least the following three endosomal biomarkers: APPL1, SORT1, and SDC1 in a biological sample from the subject as compared to a reference.

Certain embodiments of the present disclosure provide a method of detecting and measuring the severity of a prostate cancer in a subject, the method comprising detecting elevated levels of a combination of the following three endosomal biomarkers: APPL1, SORT1, and SDC1 in a biological sample from the subject as compared to a reference.

Certain embodiments of the present disclosure provide methods of monitoring the progression of a prostate cancer in a subject, the method comprising detecting one or more of an altered presence, level, secretion and distribution of at least the following three endosomal biomarkers: APPL1, SORT1, and SDC1 in a biological sample from the subject as compared to a reference.

In one embodiment, the method comprises recommending a subject for active surveillance. The subject may be one who is found to have elevated levels of APPL1 and SORT1 and a decreased level of Syndecan-1 as compared to a reference.

Certain embodiments of the present disclosure provide methods of recommending a subject for active surveillance, the method comprising detecting one or more of an altered presence, level, secretion and distribution of at least the following three endosomal biomarkers: APPL1, SORT1, and SDC1 in a biological sample from the subject as compared to a reference. The subject may be one who is found to have elevated levels of APPL1 and SORT1 and a decreased level of Syndecan-1 as compared to a reference.

Certain embodiments of the present invention provide methods of detecting a prostate cancer in a subject, the method comprising:
obtaining a biological sample from the subject;
processing the biological sample to allow detection of at least the following three endosomal markers: APPL1, SORT1, and SDC1;
detecting, in each biomarker, one or more of an altered presence, level, secretion and distribution of the selected biomarker in the processed sample; and
identifying a prostate cancer and/or the progression of the cancer in the subject.

Certain embodiments of the present invention provide methods for detecting a prostate cancer in a subject, the method comprising:
obtaining a biological sample from the subject;
processing the biological sample to allow detection of at least the following three endosomal markers: APPL1, SORT1, and SDC1;
comparing, in each biomarker, the presence, level, secretion and distribution of the selected markers; optionally with one or more other markers known to be indicative of the presence or absence of prostate cancer in the subject; and
identifying prostate cancer in the subject.

Certain embodiments of the present invention provide methods of detecting a prostate cancer in a subject, the method comprising:
processing a biological sample from said subject to allow detection of at least the following three endosomal markers: APPL1, SORT1, and SDC1;
comparing the presence, level, secretion and distribution of the selected markers; optionally with one or more other markers known to be indicative of the presence or absence of prostate cancer in the subject; and
identifying prostate cancer and/or the progression of the cancer in the subject.

Certain embodiments of the present invention provide methods of detecting a prostate cancer in a subject, the method comprising:
obtaining a biological sample from the subject;
processing the biological sample to allow detection of at least the following three endosomal markers: APPL1, SORT1, and SDC1; and
detecting one or more of an altered presence, level, secretion and distribution of these selected markers in the processed sample; and
identifying a prostate cancer and/or the progression of the cancer in the subject,
wherein the subject is effectively treated for the prostate cancer according to the biomarker pattern.

Certain embodiments of the present disclosure provide a method of screening for a prostate cancer in a subject, the method comprising detecting at least the following three endosomal markers; APPL1, SORT1, and SDC1 from a subject sample.

Certain embodiments of the present invention provide a method of screening for a prostate cancer, and determining the progression thereof, in a subject, the method comprising detecting at least the following three endosomal markers: APPL1, SORT1, and SDC1 from a subject sample, wherein one or more of an altered presence, level, secretion and distribution of the selected marker is indicative of prostate cancer and/or progression thereof in the subject.

Certain embodiments of the present invention provide a method for diagnosing (or detecting) and treating a prostate cancer in a subject, the method comprising:
detecting APPL1, SORT1, and SDC1 from the subject; and
treating the subject based on one or more of the presence, level, secretion and distribution of the selected markers detected, with a cancer therapy.

Certain embodiments of the present invention also provide a composition comprising one or more antibodies or fragment thereof that binds to at least the following three endosomal biomarkers APPL1, SORT1, and SDC1 in a biological sample obtained from a subject having prostate cancer. The one or more antibodies or fragments thereof may be bound to a detectable label. Also provided herein is a method of preparing such a composition.

Certain embodiments of the present invention provide methods of determining the likelihood of the presence of a prostate cancer in a subject, the method comprising detecting one or more of an altered presence, level, secretion and distribution of at least the following three endosomal biomarkers APPL1, SORT1, and SDC1 in a biological sample from the subject as compared to a reference.

Certain embodiments of the present invention provide methods of identifying a subject suffering from prostate cancer who is likely to be responsive to a cancer therapy, the method comprising detecting one or more of an altered presence, level, secretion and distribution of at least the following three endosomal biomarkers APPL1, SORT1, and SDC1 in a biological sample from the subject as compared a reference.

Also provided herein is a method of stratifying a subject suffering from prostate cancer into a likely responder or non-responder to a cancer therapy, the method comprising detecting elevated levels of at least the following three endosomal biomarkers APPL1, SORT1, and SDC1 in a biological sample from the subject as compared a reference.

Certain embodiments of the present invention provide methods of predicting the risk of recurrence of prostate cancer in a subject following a cancer therapy, the method comprising detecting one or more of an altered presence, level, secretion and distribution of at least the following three endosomal biomarkers APPL1, SORT1, and SDC1 in a biological sample from the subject as compared a reference.

Certain embodiments of the present invention provide for kits for performing the diagnostic/prognostic methods as described herein.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1. APPL1 defines benign tissue with intense basal cell layer staining, but shows a switch to a nuclear or cytoplasmic distribution as the cancer develops; Sortilin-1 has a polarised distribution in establishment cancer tissue; Syndecan-1 detects an advanced cancer phenotype. Examples in benign and prostate cancer tissue.

FIG. 2. Mapping the cancer with APPL1 biomarker by IHC, which reliably detects and visualises the prostate cancer to confirm diagnosis.

FIG. 3. APPL1 maps the geography of the cancer in prostatectomy patient tissue. APPL1 intensely stains the basal cell layer in benign tissue and has a quantitative increase in expression and a change in distribution to the cytoplasm of cells in establishment cancer tissue. APPL1 intensely stains advanced cancer tissue with a cytoplasmic distribution. Table shows APPL1 has very high sensitivity and specificity in detection of prostate cancer.

FIG. 4. Sortilin-1 defines establishment cancer and is detected as an intense granular staining pattern in establishment cancer, which becomes disseminated and has less expression in advanced cancer tissue. Intense granular Sotilin-1 staining of cancer tissue aligns with Gleason grade 6/ISUP grade group 1 and defines patients that should be on active surveillance (N.B. where no significant Syndecan-1 staining is present in the cancer). Table shows Sortilin-1 sensitivity and specificity in detection of prostate cancer.

FIG. 5. Syndecan-1 defines advanced cancer. Syndecan-1 has a strong staining pattern in basal cells from benign tissue (similar to APPL1), is lost in PIN tissue and becomes evident in establishment cancer as it switches to a more advanced cancer phenotype. Syndecan-1 detects migrating cancer cells and pockets of advanced cancer tissue. Table shows Syndecan-1 has high sensitivity and specificity in detection of advanced prostate cancer.

FIG. 6. Syndecan-1 defines migrating cancer cells and even small pockets of advanced cancer as seen in the top part of the figure and in an enlarged view of another small pocket of cancer in the bottom part of the figure.

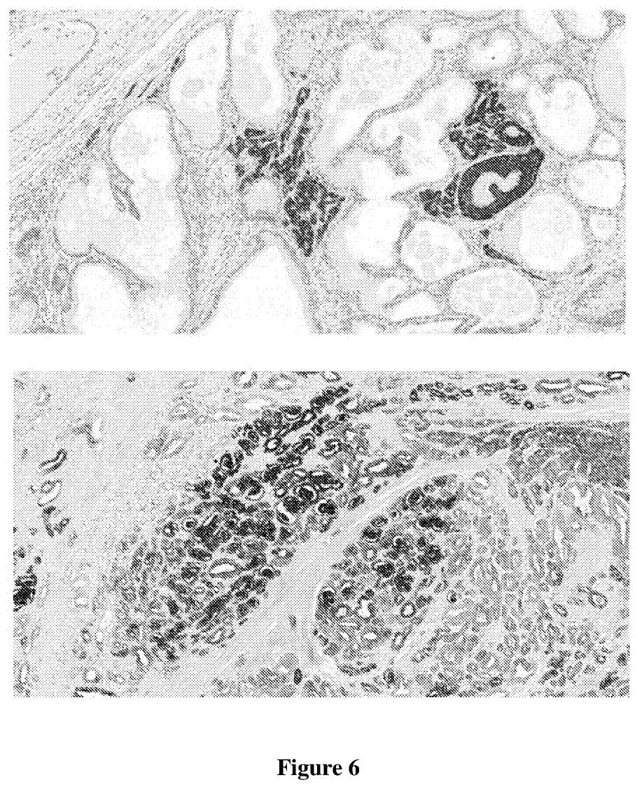

FIG. 7. APPL1 defines prostate cancer architecture to confirm diagnosis. APPL1 increases in expression in PIN tissue (arrows), has a cytoplasmic distribution in cancer tissue and has increased expression in advanced cancer compared to establishment cancer.

FIG. 8. Sortilin-1 defines establishment prostate cancer with an intense granular staining pattern, which is also evident in PIN tissue as the cancer is forming. In advanced cancer the expression of Sortilin-1 decreases and the pattern of staining is less granular and more dispersed.

FIG. 9. Syndecan-1 defines advanced prostate cancer with very intense staining pattern which is also evident in some areas of establishment cancer as it progresses to a more advanced stage.

FIG. 10. Composite pictograph depiction of potential patient management guided by APPL1, Sortilin-1 and Syndecan-1.

FIG. 11. APPL1 (EV1) and Syndecan-1 (EV3) clearly depict basal cell labelling in benign tissue and there is limited to no Sortilin-1 (EV2) staining. In clinical practice APPL1 (EV1) can be used to provide a global picture of the tissue, which in this case does not have cancer present and Sortilin-1 (EV2) and Syndecan-1 (EV3) can be used to determine, the presence of cancer or in this case benign tissue respectively, with the absence of establishment and advanced cancer.

FIG. 12. In ISUP grade group 1 patient tissue APPL1 (EV1) clearly depicts the localised cancer, with Sortilin-1 (EV2) showing intense polarised labelling and Syndecan-1 (EV3) has minimal or no staining. In clinical practice APPL1 (EV1) can be used to provide a global picture of the cancer, and Sortilin-1 (EV2) and EV3 can be used to determine the presence/absence of establishment (Sortilin-1 (EV2) positive in this patient) and advanced cancer (Syndecan-1 (EV3) negative in this case).

FIG. 13. In ISUP grade group 2 patient tissue APPL1 (EV1) marks the cancer and there are regions of Sortillin-1 (EV2) polarised labelling/Gleason grade 3, or intense Syndecan-1 (EV3) labelling/Gleason grade 4 tissue with cribriform glands. In clinical practice APPL1 (EV1) can be used to provide a global picture of the cancer, and Sortillin-1 (EV2) and Syndecan-1 (EV3) can be used to determine the presence/absence/relative amount of establishment (Sortillin-1 (EV2) positive in this patient) and advanced cancer (Syndecan-1 (EV3) positive in this case).

FIG. 14. In ISUP grade group 3 patient tissue APPL1 (EV1) maps a region of cancer with an area of Sortilin-1 (EV2 positive)/Gleason 3 labelling and a larger core of Sortilin-1 (EV2) low intensity staining and intense Syndecan-1 (EV3 positive)/Gleason 4 labelling.

FIG. 15. In ISUP grade group 4 patient tissue APPL1 (EV1) maps multiple nodes of cancer and there are typically, poorly-formed cancer glands with minimal Sortilin-1 (EV2) staining and extensive Syndecan-1 (EV3 positive staining)/Gleason grade 4 labelling in multiple areas.

FIG. 16. In ISUP grade group 5 patient tissue there is intense APPL1 (EV1) in multiple areas and nodes and intense Syndecan-1 (EV3) labelling, with minimal Sortilin-1 (EV2) labelling.

FIG. 17. In ISUP grade group 5 patient tissue there is intense APPL1 (EV1) in multiple areas and nodes and intense Syndecan-1 (EV3) labelling of cords, pockets of cancer, migrating cancer cells and regions of high intensity focal Syndecan-1 (EV3) staining.

FIG. 18. Precision biomarkers to define prostate cancer pathogenesis. The table summarises the clinical utility of the APPL1, Sortilin-1 and Syndecan-1 biomarkers.

FIG. 19. IHC using APPL1, Sortilin-1 and Syndecan-1 enables a more reliable ISUP grade grouping of prostate cancer patients (n=114 prostate cancer patients) compared to H&E ISUP grade grouping.

FIG. 20. Significant improvement in prediction of biochemical recurrence by IHC ISUP grade grouping (P=0.0002) over H&E ISUP grade grouping (P =0.001). n=144 prostate cancer patients.

FIG. 21. Significant prediction of clinical recurrence (metastasis) by IHC ISUP grade grouping, but not for H&E ISUP grade grouping. n=114 prostate cancer patients.

FIG. 22. Prediction of Clinical Recurrence with IHC. Only Prostate Cancer Patients with CR have high APPL1, low Sortilin-1, and high Syndecan-1 labelling. Patients with no Sortilin-1 present with clinical recurrence by 50 months whereas patients with small amounts of Sortilin-1 present up to 150 months. The bottom of the figure shows the APPL1 (EV1), Sortilin-1 (EV2) and Syndecan-1 (EV3) staining patterns from a typical prostate cancer patient with clinical recurrence.

FIG. 23. Patient ISUP grade grouping by H&E compared to ISUP grade grouping by IHC with Sortilin-1 (EV2) and Syndecan-1 (EV3). The green boxes show consensus cases with pathology that is clearly recognised by both technologies; the white boxes to the left of the green boxes show prostate cancer patient cases that were over reported based on H&E or under reported on the right hand side of the green boxes. These changes in interpretation result in the improved reliability (FIG. 19) and increased capacity to recognise biochemical recurrence (BCR, FIG. 20) and clinical recurrence (CR, FIG. 21).

FIG. 24. Validated APPL1 immunoassay for the detection of prostate cancer in patient plasma samples. APPL1 shows significant separation of prostate cancer and control human plasma samples.

FIG. 25. Schematic showing the structure of Syndecan-1 with protein core and potential proteolytic clipping sites and carbohydrate side chains.

FIG. 26. Comparison of IHC for commercially available antibodies to Syndecan-1 and Envision Sciences monoclonal antibodies to Syndecan-1.

FIG. 27. Comparison of APPL1, Sortillin-1 and Syndecan-1 IHC in needle core biopsies and prostatectomy sections from prostate cancer patients, demonstrating equivalent detection of pathogenesis in both tissue samples.

FIG. 28. An example of using APPL1, Sortillin-1 and Syndecan-1 IHC to determine whether a prostate cancer patient is suitable for Active Surveillance.

FIG. 29. Comparison of Envision Sciences monoclonal antibodies to Sortillin-1 showing that only SEQ ID NO: 4 accurately depicts the pathogenesis in prostate cancer patient tissue samples. Anti-Sortilin-1 mouse monoclonal antibodies were generated (Genscript, Piscataway, NJ 08854, USA.) using the peptide sequence WVSKNFGGKWEEIHK (SEQ ID NO: 4) and EKDYTIWLAHSTDPE (SEQ ID NO: 5).

### DETAILED DESCRIPTION

The present disclosure is based on the determination that the combination of at least three specifically identified endosomal markers can be used for the detection, diagnosis and prognosis of prostate cancer, in particular can be used as a determination of the progression and severity of the cancer in a subject and will therefore aid in the physicians recommended course of treatment.

Certain embodiments of the present disclosure provide methods for detecting a prostate cancer in a subject. Certain embodiments of the present disclosure provide methods for determining the progression of a prostate cancer in a subject. Certain embodiments of the present disclosure provide methods of treating prostate cancer in a subject based on a diagnosis and prognosis of at least the three selected endosomal markers, specifically APPL1 (Adaptor protein, phosphotyrosine interacting with PH domain and leucine zipper 1, APPL1), Sortilin-1 (SORT1), and Syndecan-1 (SDC1). Other embodiments are disclosed herein.

Certain embodiments of the present disclosure provide methods of detecting a prostate cancer in a subject, the method comprising detecting one or more of an altered presence, level, secretion and distribution of at least the following three endosomal biomarkers APPL1 (Adaptor protein, phosphotyrosine interacting with PH domain and leucine zipper 1, APPL1), Sortilin-1 (SORT1), and Syndecan-1 (SDC1) in a biological sample from the subject as compared to a reference.

Certain disclosed embodiments have one or more combinations of advantages. For example, some of the advantages of the embodiments disclosed herein include one or more of the following: the identification of a new class of specific three markers which work interactively for the diagnosis and/or prognosis of prostate cancer; and additional one or more markers that in some instances may be additionally useful for both diagnosis and prognosis of prostate cancer.

These markers may be readily detectable in a biological sample, such as in a tissue samples, or tissue biopsies, or blood/plasma samples or in urine samples.

The utility/advantages of the three biomarkers APPL1, Sortilin-1 and Syndecan-1 for cancer detection and grading in tissue samples or tissue biopsies:—
The combination of APPL1 and Syndecan-1 distribution can be used to define the onset of cancer (PIN tissue formation). APPL1 undergoes a distribution change from clearly defined basal cell staining to a cytoplasmic distribution in PIN tissue, often with the staining of nuclear inclusions in selected cells. Syndecan-1 has a very intense basal cell staining pattern in benign tissue, but is lost as PIN tissue forms.
This combination of APPL1 and Syndecan-1 effectively identifies prostate cancer initiation in tissue/biopsy samples. APPL1 increases in tissue expression with the formation of establishment cancer and Sortilin-1 exhibits a specific polarised distribution in establishment cancer. This combination of biomarkers identifies patients with Gleason score <6 (Gleason grade/GG 3+3) cancers or ISUP grade group 1 and at this stage the cancer will have minimal to no Syndecan-1 staining.
This combination of the three biomarkers effectively identifies an early stage of cancer progression. APPL1 is optimal for scanning large areas of tissue to map the cancer and identify the cancer and its boundaries, and the stroma is not stained (no background); but it is difficult to distinguish whether glands are cribriform/fused glands, compared to closely arranged Gleason grade 3 glands. Syndecan-1 provides confirmation for this staining and is frequently distributed in the same manner as APPL1 even though it is more distinct and granular. However, Sortilin-1 clearly distinguished between cribriform and fused glands in tissue/biopsy samples. Sortilin-1 is optimal for this visualisation because the staining is polar, and the cells and borders are better distinguished, demonstrating clearly separated Gleason grade 3 glands compared to fused/cribriform glands. In Gleason grade 4/5 cancers where cells proliferate to form fused glands/sheets, APPL1 cannot differentiate the fused glands versus sheets of cells, whereas Syndecan-1 indicates the presence of fused glands and combined with Sortilin-1 confirms this morphology. Because APPL1 stains the stroma and appears more sheet like in all of the tissue samples/biopsy cores, whereas Sortilin-1 and Syndecan-1 do not, the spaces (indicating fused glands) or stroma (indicating sheets) are clearly distinguished with this combination of biomarkers. While there is considerable co-staining of the three biomarkers, higher amounts of APPL1 and Syndecan-1 with reduced Sortilin-1 signifies advanced cancer; the specific pattern of intense APPL1 staining and intense Syndecan-1 staining, but with little or no Sortilin-1 staining is observed for patients at high risk of clinical recurrence (metastasis). The combination of APPL1 (due to its increasing intensity as the cancer progresses to an advanced stage) and Syndecan-1 (due to its high intensity staining in advanced cancer and in migrating cancer cells) enables the effective detection of advanced cancer; this is balanced against Sortilin-1 expression to make decisions on how advanced the cancer is. The combination of the three biomarkers APPL1, Sortilin-1 and Syndecan-1 enables:—
   More accurate detection of cancer, facilitating optimal identification of the cancer in patients previously graded Gleason score <6 or ISUP grade group 1. This is particularly important for assessing patients who are suitable for active surveillance where patients should only be Gleason score <6 or ISUP grade group 1; this can be identified by APPL1 and Sortilin-1 staining without any signs of Syndecan-1 staining (i.e. Syndecan-1 accurately identifies even small pockets of cancer and signifies an advanced cancer phenotype).
   The combination and relative proportions of Sortilin-1 and Syndecan-1 define how advanced the cancer is and can be used to more reliably define ISUP grade groups 1-5 (with Sortilin-1 only for ISUP-1 and increasing amounts of Syndecan-1 for the other grade groupings; and intensity of APPL1 staining to confirm progression).
   Detection of the presence of cancer at a distance from the primary pathogenesis; i.e. signs of the cancer at a distance from the primary pathology or origin of the cancer
   More definitive identification of advanced cancer in Gleason score 7 (GG 3+4 or 4+3) or ISUP grade group 2 and 3 patients
   Specific recognition of patients at risk of clinical recurrence.
The optimal detection of the three biomarkers APPL1, Syndecan-1 and Sortilin-1 in prostate cancer patient tissue involves the production of monoclonal antibodies to specific linear sequences on each of the proteins:—
   Specific linear sequence from the APPL1 protein involving but not limited to: SRLIAASSRPNQASSEGQFVVL (SEQ ID NO: 9)
   Specific linear sequence from the Sortilin-1 protein involving but not limited to: ENGLWVSKNFGGKWEEIHKA (SEQ ID NO: 10)
   Specific linear sequence from the Syndecan-1 protein involving but not limited to: EPKQANGGAYQKPTKQEEFYA (SEQ ID NO: 11).

The methods herein may be amenable to high throughput analysis of samples.

Further, certain embodiments of the present disclosure are based, at least in part, on the recognition that a unique change in the cell biology of endosomes occurs in prostate cancer cells and tissue. This change involves cell surface proteins that are specifically internalised into early endosomes and involves early endosome and recycling endosome vesicular machinery. The expression of specific proteins in this pathway have the capacity to accurately depict prostate cancer pathogenesis and three specific biomarkers have been selected from a panel of >20 biomarkers for this specific purpose. Because of the inter-related biology and different functional properties of these three biomarkers, they need to be used in concert to fully depict the pathogenesis and to inform about cancer progression and prognosis. This provides a unique set of changes to these endosome related biomarkers in prostate cancer cells/tissue and with a combination of these three biomarkers, effectively identifies critical changes in prostate cancer tissue when compared to control tissue. The high sensitivity and specificity of these biomarkers for prostate cancer, especially when used in combination (≥95%) is the first set of biomarkers to provide this level of definitive visualisation. The detection of these three endosomal biomarkers in other patient samples including blood and urine is implied by the known biology of these proteins and has been demonstrated by release from prostate cancer cells in vitro. In addition, a ratio of the three biomarkers provides a risk assessment for the onset of clinical recurrence/metastasis in patients, enabling specific advice on therapeutic intervention.

The biology of each of the three biomarkers APPL1, Sortilin-1 and Syndecan-1 is intimately connected to the pathogenic process and represents key control points for other biology. APPL1 is a transcription factor that is also involved in endosome traffic and recycling and controls growth factor uptake and signaling. The transcription factor activity of APPL1 is evident particularly in PIN tissue where it stains a significant proportion (20-30%) of nuclei, and is presumably involved in regulating gene expression.

Sortilin-1 is a key molecule in GLUT4 vesicle biogenesis and also interacts with GLUT1 to concertedly regulate sugar metabolism and the Warburg effect. All elements of the GLUT4 vesicle biogenesis and trafficking processes are androgen regulated (e.g. AS160. Sortilin-1 also binds and regulates lipoprotein lipase (LPL), oxy sterol binding protein (OSBP) and progranulin (PGRN/GRN) and when downregulated this releases these ligand proteins to drive advanced cancer, which involves lipid metabolism, angiogenesis, and Syndecan-1 biology. Syndecan-1 potentiates the advanced signaling and growth factor biology and also binds beta3 integrins, which is thought to drive platelet interaction and immune cloaking; and also activates Survivin to limit apoptosis. Syndecan-1 engages extracellular matrix molecules like fibronectin (FN1), vitronectin (VTN), laminins and collagens through its heparin and chondroitin sulphate side chains, playing a role in attachment spreading and tissue invasion. The three key control points for prostate cancer pathogenesis are therefore depicted by APPL1, Sortilin-1 and Syndecan-1 and are representative of the wider pathogenic process, which involves the other biomarkers described above, which are involved in sugar and lipid metabolism together with inflammation, migration, signaling, immune cloaking and angiogenesis. In certain embodiments the additional biomarkers listed above are contemplated as surrogates of APPL1, Sortilin-1 and Syndecan-1.

In one embodiment, the methods as defined herein may include detecting an APPL1 protein, a SORT1 protein and a SDC1 protein. The methods may include detecting a variant of APPL1 protein, a variant of SORT1 protein and/or a variant of SDC1 protein.

In one embodiment, the methods as defined herein may include detecting an APPL1 nucleic acid, a SORT1 nucleic acid and a SDC1 protein nucleic acid. The nucleic acid may be an mRNA.

Certain embodiments of the present disclosure provide methods of detecting a prostate cancer in a subject, the method comprising detecting elevated levels of at least the following three endosomal biomarkers APPL1, SORT1, and SDC1 in a biological sample from the subject as compared to a reference.

Certain embodiments of the present disclosure provide methods of detecting and measuring the severity of a prostate cancer in a subject, the method comprising detecting one or more of an altered presence, level, secretion and distribution of at least the following three endosomal biomarkers: APPL1, SORT1, and SDC1 in a biological sample from the subject as compared to a reference.

The method may, for example, be able to detect a prostate cancer and distinguish between a prostatic intraepithelial neoplasia (PIN), a primary (or establishment) prostate cancer, and a metastatic prostate cancer to determine the severity of the prostate cancer. The method may, for example, distinguish a metastatic prostate cancer from PIN and a primary prostate cancer.

Certain embodiments of the present disclosure provide a method of detecting and measuring the severity of a prostate cancer in a subject, the method comprising detecting elevated levels of a combination of the following three endosomal biomarkers: APPL1, SORT1, and SDC1 in a biological sample from the subject as compared to a reference.

Certain embodiments of the present disclosure provide methods of monitoring the progression of a prostate cancer in a subject, the method comprising detecting one or more of an altered presence, level, secretion and distribution of at least the following three endosomal biomarkers: APPL1, SORT1, and SDC1 in a biological sample from the subject as compared to a reference.

The method may, for example, be able to distinguish between a prostatic intraepithelial neoplasia (PIN), a primary prostate cancer, and a metastatic prostate cancer to determine the progression of the prostate cancer.

In some embodiments, the method comprises detecting an APPL1 polypeptide comprising the amino acid sequence of SEQ ID NO: 1, SEQ ID NO: 2 and/or SEQ ID NO: 3.

In some embodiments, the method comprises detecting an SORT1 polypeptide comprising the amino acid sequence of SEQ ID NO: 4.

In some embodiments, the method comprises detecting an SDC1 polypeptide comprising the amino acid sequence of SEQ ID NO: 6 or 8.

In certain embodiments, the prostate cancer is selected from a prostatic intraepithelial neoplasia (PIN), a primary prostate cancer, and a metastatic prostate cancer. Other forms and/or grades of prostate cancer are contemplated. The present invention is able to provide a differentiation of such cancers to aid the physician to recommend a beneficial and targeted therapy for improved outcomes for the patient. PIN is defined as neoplastic growth of epithelial cells within pre-existing benign prostatic acini or ducts (i.e has progressed beyond hyperplasia). We understand that PIN satisfies many of the requirements for the event of transformation from a pre-malignant condition to a cancer (hyperplasia to neoplasia transformation). In addition, high-grade PIN (HGPIN) is more widely accepted as having transited to prostate cancer neoplasia/malignancy or is formed as cancer progresses through ducts or into other tissue. HGPIN can be visualised with the biomarkers in prostate cancer patient tissue as “Roman bridges” in the ducts or with very strong APPL1/Syndecan-1 staining in ductal and tissue regions. The biomarkers therefore identify the transition from hyperplasia to neoplasia/prostate cancer.

In this regard, typically the Gleason Grading system is currently used to evaluate a prostate cancer. A “score” is assigned to a prostate cancer on the basis of the combination of a “Gleason” pattern associated with various features of a tumour specimen and a subsequent grade assigned to the patterns of the tumour specimen. A Gleason score of 2-6 is considered to be a cancer of low aggressiveness and is assigned to ISUP grade group 1. In these patients where the cancer is confined to the prostate patients can be recommended for active surveillance. Different stages of advanced cancer are defined as: A Gleason score of 7 is considered to be intermediate to moderate aggressiveness and is now divided into ISUP 2 (GG 3+4) and ISUP 3 (GG 4+3) grade groups; A Gleason score of 8-10 or ISUP 4/5 is considered to be a cancer of high aggressiveness. In certain embodiments, the prostate cancer is a cancer with a Gleason score or ISUP grade grouping of any of the aforementioned scores and these scores may be more accurately defined with biomarkers than standard H&E histology.

There are currently very significant problems with Gleason grading/ISUP grade grouping and reliability by H&E histology: 1) A significant number of Gleason score <6 or ISUP grade group 1 patients have advanced cancer; 2) Patients with Gleason score 7 or ISUP group 2 and 3 patients have variable outcomes and it is not easy to recognise which patients require active intervention and active surveillance/watchful waiting; 3) Sampling error or missing the cancer with the biopsy procedure, makes it difficult to confirm diagnosis and perform grading/predict prognosis and repeat biopsies are common; 4) When patients are detected early and the cancer is small and confined (stage 1 as described below) it is more difficult to define a Gleason grade; 5) It is not possible to accurately predict which patients will have clinical recurrence/metastasis using Gleason grading or ISUP grade grouping. The advantage of our technology is that we have a set of three biomarkers that enable precise detection of the cancer, and can distinguish establishment and advanced cancer, which can consequently facilitate more accurate Gleason grading/ISUP grade grouping, provide optimal detection of advanced cancer and can identify patients at risk of clinical recurrence.

The “Epstein” grading system is discussed in Epstein J I, Zelefsky M J, Sjoberg D D, et al. A contemporary prostate cancer grading system: A validated alternative to the Gleason score, Eur Urol (2015)—enclosed herein by reference; and agreed to by the International Society of Urological Pathology to be used as an ISUP grade grouping.

As discussed above the problems with the current Gleason grading system can be summarised as follows:
1. Gleason Scores 2-6 are currently no longer assigned (now ISUP grade group 1) and certain patterns that were defined as Gleason score of 6 are now graded as 7 (ISUP grade group 2), thus leading to contemporary Gleason score 6 cancers having a better prognosis than historic score 6 cancers.
2. The combination of Gleason scores into a 3-tier grouping (6,7,8-10) is used for prognostic and therapeutic purposes, despite 3+4=7 vs. 4+3=7 and 8 vs. 9-10 having very different prognoses. In addition these are now assigned ISUP grade groups 2-5 but while this has increased reliability somewhat it is still not ideal based on the ability to define advanced cancer with H&E, which is not a cancer specific stain, just a morphological dye.
3. In practice the lowest Gleason score is now assigned a 6 or IUSP grade group 1, even though it is on a scale of 1-5 for both the main and secondary area of cancer. This leads to a logical yet incorrect assumption on the part of patients that their cancer is in the middle of the scale, compounding the fear of their cancer diagnosis with the belief that the cancer is serious, thus leading to an expectation that treatment is necessary.

To partially address the above deficiencies with Gleason grading and Gleason scoring, the ISUP 5 tier Grade Group system has been developed based on a study of >20,000 prostate cancer cases treated with radical prostatectomy and >5,000 cases treated by radiation therapy (see composite photograph for different patterns).
Grade Group 1 (Gleason score ≤6)—Only individual discrete well-formed glands
Grade Group 2 (Gleason score 3+4=7)—Predominantly well-formed glands with a lesser component of poorly-formed/fused/cribriform glands
Grade Group 3 (Gleason score 4+3=7)—Predominantly poorly-formed/fused/cribriform glands with a lesser component of well-formed glands††For cases with >95% poorly-formed/fused/cribriform glands or lack of glands on a core or at RP, the component of <5% well-formed glands is not factored into the grade
Grade Group 4 (Gleason score 8)
   Only poorly-formed/fused/cribriform glands or
   Predominantly well-formed glands with a lesser component lacking glands††or ††Poorly-formed/fused/cribriform glands can also be a more minor component
   Predominantly lacking glands with a lesser component of well-formed glands††††Poorly-formed/fused/cribriform glands can also be a more minor component
Grade Group 5 (Gleason scores 9-10)—Lacks gland formation (or with necrosis) with or w/o poorly-formed/fused/cribriform glands††For cases with >95% poorly-formed/fused/cribriform glands or lack of glands on a core or at RP, the component of <5% well-formed glands is not factored into the grade
1. The five-year biochemical recurrence (BCR)-free progression probabilities for radical prostatectomy ISUP Grade Groups 1-5 were 96%, 88%, 63%, 48%, and 26%; which respectively means BCR still occurs in respectively 4%,12%, 37%, 52%, and 74% of these grade groups. In addition, these grade grouping figures do not define Clinical recurrence/metastasis and only relate to BCR.
2. The 5 ISUP Grade Groups were also predictive for biopsy grade followed by radical prostatectomy or radiation therapy.
3. The new system distils grades of prostate cancer down to the lowest number of grades, each with a unique prognosis, but there are still problems with reliability and interpretation. However, as a result of significant differences in criteria and reporting compared to the Gleason's original grading system, we have regarded the newly proposed grade groups as a new grading system.

Although currently being adopted the ISUP grade grouping system (Also referred to as Epstein grading system in parts of US/Europe) has the following advantages:
1. More accurate grade stratification than the traditional Gleason scoring system
2. Simplified grading system of 5 as opposed to multiple possible scores depending on various Gleason pattern combinations
3. Lowest grade is 1 as opposed to current practice of Gleason score 6, with the potential to reduce overtreatment of indolent prostate cancer
The new grading system, using the above terminology, has been accepted by the 2016 World Health Organization (WHO).

Prostate cancers may also be categorised by stage, being a measure of how far a cancer has developed or has been contained within the prostate. In Stage 1, the cancer is focal and contained within the prostate. In Stage 2, the cancer is larger and may be in both lobes of the prostate, but is still confined to the organ. In Stage 3, the cancer has spread beyond the prostate (e.g. broken the capsule or extended down the ducts of the prostate) and may have invaded the adjacent lymph glands or seminal vesicles. In Stage 4, the cancer has spread to other organs, or to bone, which is referred to as metastasis. In certain embodiments, the prostate cancer is a cancer with a staging of any of the aforementioned stages. The advantage of our biomarkers is that they provide optimal detection of the cancer and provide improved visualisation of the cancer (both establishment and advanced cancer) compared to H&E staining, which enables better visualisation of the cancer location, spread and pathogenesis.

It will be appreciated that while the present disclosure is described with reference to detecting a prostate cancer in a human subject, the present disclosure contemplates detecting prostate cancer in an animal subject, and accordingly veterinary applications of the present disclosure are also contemplated.

In certain embodiments, the subject is suffering from a prostate cancer. Examples of prostate cancers are as described herein.

In certain embodiments, the subject is a subject with an increased likelihood or risk of suffering from a prostate cancer. In certain embodiments, the subject is a subject susceptible to a prostate cancer. In certain embodiments, the subject is a subject with one or more risk factors associated with a prostate cancer. In certain embodiments, the subject is a subject with an unknown likelihood or risk of suffering prostate cancer.

In certain embodiments, the subject is a subject with a measured or known PSA level. Examples of PSA levels are as described herein, for example, as described in Example 6 herein. In certain embodiments the subject is a subject with one or more of the characteristics as described in one or more of the Figures and/or Examples.

The term “associated marker” refers to an optional other biomarker which is enriched in one or more particular tissues, cells, organelles, and/or cell compartments and as such can be used with other markers, to further assist in the identification of the tissue, cell, organelle, and/or cell compartment.

The selected biomarker comprises at least the following three: APPL1, Sortilin-1 and Syndecan-1, and/or a gene/mRNA encoding one of the aforementioned proteins, a fragment of one of the aforementioned proteins/genes/mRNA, a derivative of one of the aforementioned proteins/genes/mRNA, and a processed form of one of the aforementioned proteins/genes/mRNA. Other related biomarkers are contemplated that interact with the key proteins APPL1 (e.g. APPL2, Rab5, EGFR, Rab21, OCRL, GIPCL myosin VI), Sortilin-1 (e.g. GLUT-4, GLUT-1, LPL, OSBP, PGRN, NTS, RAP), Syndecan-1 (e.g. Syntenin-1, Survivin, ITGB3, ITGB5, PDGF, IGF1R, VEGF's, FN1, VTN, lamanins, BMP's, PAI-1, FGF's).

It will be appreciated that the selected markers of the present disclosure are referred to herein as the human forms of the selected markers. However, it will be appreciated that the detection and/or use of equivalent or synergistic markers are also contemplated. Methods for detecting markers are known in the art. Typically, a marker present in a subject is detected in a sample, or a processed form of a sample, taken from a subject. For example, methods for detecting proteins and RNAs are known and may be performed typically using commercially available products. General methods, including methods for protein and RNA detection, extraction and isolation are known, are as described in, for example, Ausubel et al., Current Protocols of Molecular Biology, John Wiley and Sons (1997), the entire contents of which is hereby incorporated by reference.

Methods for detection of proteins markers are known and include for example immunological detection methods such as immunobinding, immunoblotting (e.g., Western analysis), immunoprecipitation, immunoelectrophoresis, immunostaining, immunohistochemistry, spectrophotometry, enzyme assays, mass spectrometry, and microscopy. Other methods are contemplated.

Methods for detecting nucleic acids are known and include microarray analysis, blotting (Northern, Southern), in situ hybridization, RT-PCR , End-Point Stem-Loop Real-Time RT-PCR, miR-Q RT-PCR, (A)-Tailed Universal Reverse Transcription, RNA Amplification Profiling, cloning based methods, nanoparticle based methods, splinted ligation methods, padlock-probes and rolling-circle amplification, bead-based flow cytometric methods, bioluminescence RNA detection methods, molecular beacon methods, ribozyme methods, and quantitative LNA-ELF-FISH methods. Other methods are contemplated.

In certain embodiments, the detecting of RNA markers comprises reverse transcription. Methods for reverse transcribing RNAs are known in the art. In certain embodiments, the detecting of RNA markers comprises amplification of a nucleic acid. Methods for nucleic acid amplification are known in the art. In certain embodiments, the detecting of RNA markers comprises a polymerase chain reaction. In certain embodiments, the polymerase chain reaction comprises a quantitative polymerase chain reaction.

In certain embodiments, the detecting of RNA markers comprises binding or hybridization of nucleic acids to one or more target nucleic acids. In certain embodiments, the detecting of RNA markers comprises binding of nucleic acids to one or more target nucleic acids bound to a solid substrate, such as a chip. Methods for binding nucleic acids to target nucleic acids, including binding to nucleic acids bound to a solid substrate, are known.

In certain embodiments, the detecting of the selected marker comprises a polymerase chain reaction. In certain embodiments, the polymerase chain reaction comprises a quantitative polymerase chain reaction.

In certain embodiments, the detecting of the selected marker comprises immunological detection. In certain embodiments, the immunological detection comprises ELISA or immunoassay, staining with an antibody, immunohistochemistry, and/or flow cytometric detection. Methods involving immunological detection are known in the art.

In certain embodiments, the methods as described herein comprise detecting one or more of the presence, level, expression, secretion and distribution of the selected marker.

In certain embodiments, one or more of an altered presence, altered level, altered expression, altered secretion and altered distribution of the selected marker is indicative of a prostate cancer in the subject.

In certain embodiments, an increased level and/or an increased secretion of an endosomal marker is indicative of prostate cancer in the subject. In certain embodiments, a decreased level and/or a decreased secretion of an endosomal marker is indicative of prostate cancer in the subject.

In certain embodiments, the endosomal biomarkers are proteins or nucleic acids (such as mRNAs).

In certain embodiments, an increased level of a mRNA marker is indicative of prostate cancer in the subject. In certain embodiments, a decreased level of a mRNA marker is indicative of prostate cancer in the subject.

In certain embodiments, an increased level and/or an increased secretion of an endosomal marker is indicative of prostate cancer in the subject.

In certain embodiments, the method detects an increased level of APPL1 protein and/or mRNA, an increased secretion of APPL1 protein, an increase in secretion of Sortilin-1 protein and/or mRNA, and an increase in Syndecan-1 protein and/or mRNA. In certain embodiments, the method detects a low or high level of APPL1 protein, high or low level of Sortilin-1 and a low or high level of Syndecan-1, and/or mRNA, which is indicative of prostate cancer in the subject. All combinations of these three biomarkers are contemplated to define the pathogenic process in prostate cancer.

In certain embodiments, an altered presence, altered level, altered expression, altered secretion and altered distribution of one or more markers is as compared to one or more of non-malignant tissue, prostatic intraepithelial neoplasia, primary prostate cancer and metastatic prostate cancer.

In certain embodiments, an increase in APPL1 level, an increase or decrease in Sortilin-1 level and an increase in Syndecan-1 level, as compared to a reference is indicative of prostate cancer in a subject. In certain embodiments, an increase in APPL1, Sortilin-1 and Syndecan-1 levels, as compared to a reference, is indicative of prostate cancer in a subject.

In certain embodiments, an increase in APPL1, Sortilin-1 and Syndecan-1 levels, as compared to a reference, is indicative of establishment prostate cancer in a subject.

In certain embodiments, an increase in APPL1 level, a decrease in Sortilin-1 level and an increase in Syndecan-1 level, as compared to a reference, is indicative of advanced prostate cancer (or metastatic prostate cancer) in a subject. In one embodiment, the method as defined herein may distinguish a metastatic prostate cancer from a non-metastatic prostate cancer.

In certain embodiments, APPL1 protein or mRNA is increased in primary prostate cancer as compared to non-malignant tissue, Sortilin-1 protein or mRNA is increased or decreased in primary prostate cancer as compared to non-malignant tissue, and Syndecan-1 protein or mRNA is increased in primary prostate cancer as compared to non-malignant tissue.

In certain embodiments, APPL1 protein or mRNA is increased, Sortilin-1 protein or mRNA is decreased and Syndecan-1 protein or mRNA is increased in metastatic prostate cancer as compared to primary or establishment prostate cancer.

In certain embodiments, an altered distribution of APPL1 as compared to a reference indicates the presence of prostate cancer (such as establishment prostate cancer or advanced prostate cancer) in a subject. The altered distribution may, for example, be a change from a basal cell staining in a reference to cytoplasmic distribution with staining of nuclear inclusions in a prostate cancer sample.

In certain embodiments, an altered distribution of Syndecan-1 as compared to a reference indicates the presence of prostate cancer (such as prostatic intraepithelial neoplasia (PIN), a primary prostate cancer or a metastatic prostate cancer) in a subject. The altered distribution may, for example, be a change from basal cell staining pattern in a reference to the loss of the basal cell staining pattern in a prostate cancer sample.

In certain embodiments, a granular staining pattern in Sortilin-1 indicates the presence of prostatic intraepithelial neoplasia (PIN) or establishment prostate cancer. In one embodiment, a decrease in the level of Sortilin-1 as compared to a reference indicates the presence of an advanced cancer. In one embodiment, an altered distribution of Sortilin-1 indicates the presence of an advanced cancer. The altered distribution may, for example, be a less granular and more dispersed pattern of staining.

The term “elevated level” may refer to an increase in level of at least 1.1 times, 1.2 times, 1.3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times,93 times, 94 times, 95 times, 96 times, 97 times, 98 times, 99 times or 100 times or anywhere in between as compared to a reference.

Certain embodiments of the present invention provide methods of determining the likelihood of the presence of a prostate cancer in a subject, the method comprising detecting one or more of an altered presence, level, secretion and distribution of at least the following three endosomal biomarkers APPL1, SORT1, and SDC1 in a biological sample from the subject as compared to a reference.

The phrase “likelihood of the presence of a prostate cancer” refers to how likely it is for a prostate cancer to be present in a subject. An elevated levels of at least a group of the three endosomal biomarkers APPL1, Sortilin-1 and Syndecan-1 indicate a likelihood (i.e. chance or risk) of the presence of prostate cancer in the subject. This could be, for example, a more than 10%, 20%, 30%, 40%, 50%, 60%, 70% 80%, 90% or 99% likelihood of the presence of prostate cancer in the subject.

In certain embodiments, the methods as described herein comprise obtaining a biological sample from the subject.

In certain embodiments, the methods as described herein comprise processing the biological sample to allow detection of the selected marker. In certain embodiments, the methods as described herein comprise processing a biological sample to allow detection of a marker as described herein and detecting the marker in the processed sample. In certain embodiments, the methods as described herein comprise obtaining a biological sample from the subject and processing the biological sample to allow detection of the selected marker.

The term “biological sample” refers to a sample obtained from the subject and/or a processed and/or treated form thereof. For example, the biological sample may be untreated, diluted, a derivative, an extract, a treated form, pre-cleared, filtered, desalted, concentrated, diluted, buffered, centrifuged, induced, pre-treated, processed to remove one or more components or impurities from the sample, sliced, fixed, adhered to a slide, or suitable combinations thereof. In certain embodiments, a selected marker is detected in the sample directly. In certain embodiments, a selected marker is detected in the sample after processing and/or treating. In certain embodiments, the sample is processed and/or treated prior to detecting the selected marker and/or concurrently with detecting the selected marker.

Examples of biological samples include one or more biological fluids, such as blood, plasma, urine, amniotic fluid, tears, saliva, hair, skin, and one or more tissue samples or a biopsy. Other types of biological samples are contemplated.

In certain embodiments, the biological sample comprises one or more of a blood sample, a plasma sample, a serum sample, a biopsy and a prostate tissue sample.

In certain embodiments, the biological sample comprises a biopsy or a tissue sample.

Certain embodiments provide detecting the in situ level of a selected marker.

In certain embodiments, the selected marker comprises one or more blood markers, plasma markers, and/or serum markers. Certain embodiments provide detecting the circulating level of a selected marker.

In certain embodiments, the detecting comprises a qualitative determination. In certain embodiments, the detecting comprises a qualitative determination of whether the selected marker has one or more of an altered presence, an altered level, an altered expression, an altered secretion and an altered distribution. In certain embodiments, the detecting comprises a quantitative determination of whether the selected marker has one or more of an altered presence, an altered level, an altered expression, an altered secretion and an altered distribution.

In certain embodiments, the detecting comprises a qualitative determination. In certain embodiments, the detecting comprises a qualitative determination of whether the selected marker is present or absent. In certain embodiments, the detecting comprises a quantitative assessment of the level of the selected marker. For example, certain methods allow for the quantification of the concentration of the selected marker. Methods for the calculation or determination of the concentration of markers are known in the art.

Certain embodiments of the present disclosure comprise detecting two or more selected biomarkers. Certain embodiments of the present disclosure comprise detecting three or more selected biomarkers. Certain embodiments of the present disclosure comprise detecting four or more selected biomarkers.

In certain embodiments, the methods of the present disclosure comprise detecting two or more of the selected biomarkers. In certain embodiments, the methods comprise detecting three or more of the selected biomarkers. In certain embodiments, the methods comprise detecting four or more of the selected biomarkers.

In certain embodiments, the methods as described herein comprise determining the ratio of the level of one selected marker to another selected marker.

In certain embodiments, an altered ratio of biomarkers is indicative of a prostate cancer in the subject. In certain embodiments, an altered ratio as compared to non-malignant tissue is indicative of a prostate cancer in the subject. Other forms of comparison between different types of prostate tissue are as described herein.

In certain embodiments, altered ratios of APPL1, Sortilin-1 and Syndecan-1 biomarkers compared to non-malignant tissue is indicative of a prostate cancer in the subject. In certain embodiments, an increased ratio of APPL1 and Syndecan-1 compared to Sortilin-1 is indicative of advanced prostate cancer in the subject.

In certain embodiments, the methods of the present disclosure comprise detecting one or more other biomarkers in addition to the selected biomarker(s).

In certain embodiments, the methods of the present disclosure provide use of one or more biomarkers, control biomarkers and/or reference markers, as described herein.

In one embodiment, the reference is a normal or benign prostate tissue or a normal blood or plasma sample. The normal or benign prostate tissue may be one that is from the same subject or a different subject (i.e. a healthy subject). The normal blood or plasma sample may be one from a different subject (i.e. a healthy subject). The reference may also be a cut-off value (such as a predetermined cut-off value).

An alteration in the presence, level, expression, secretion and distribution of a biomarker is typically relative to the level of one or more corresponding biomarkers, for example one or more corresponding proteins or mRNAs in one or more control subjects and/or one or more subjects known to have prostate cancer.

In certain embodiments, the methods as described herein comprise comparing the presence, level, expression, secretion and distribution of the selected biomarker with one or more other biomarkers known to be indicative of a prostate cancer in a subject and/or known to be indicative of the absence of a cancer.

In certain embodiments, the methods as described herein comprise comparing the presence, level, expression, secretion and distribution of the selected biomarker to one or more reference and/or control biomarkers.

In certain embodiments, the methods as described herein comprise comparing the presence and/or level of the selected biomarker with the presence and/or level of one or more other biomarkers associated with an altered risk of prostate cancer and/or one or more other biomarkers known to be indicative of the presence or absence of prostate cancer in the subject.

In certain embodiments, the reference biomarker comprises an endogenous marker. In certain embodiments, the reference marker comprises an exogenous biomarker. For example, a sample may be spiked with an exogenous reference biomarker.

In certain embodiments, the methods of the present disclosure comprise processing the biological sample to allow detection of the selected biomarkers. In certain embodiments, the methods of the present disclosure comprise processing a biological sample obtained from the subject to allow detection of the selected biomarker. Subjects are as described herein.

In certain embodiments, the methods and kits as described herein comprise use of one or more reagents for processing a sample for analysis.

In certain embodiments, the methods as described herein further comprise obtaining information relating to one or more clinical characteristics of the subject and using the information in combination with one or more of the presence, level, secretion and distribution of the selected marker to detect prostate cancer in the subject. In certain embodiments, the one or more clinical characteristics comprise one or more of age, body mass index, smoking, genetics and family history of cancer and/or prostate cancer.

In certain embodiments, the methods as described herein further comprise obtaining information relating to one or more clinical characteristics of the subject and using the information in combination with one or more of the presence, level, expression secretion and distribution of the selected marker to detect prostate cancer in the subject or the absence of prostate cancer.

In certain embodiments, the methods as described herein comprise using a computer processor means to process data associated with one or more of the presence, level, secretion and distribution of the selected marker to generate a likelihood and/or risk of the presence of prostate cancer in the subject. Examples of computer processor means are known.

In certain embodiments, the methods have a sensitivity of detection of 0.60 or greater. In certain embodiments, the methods have a sensitivity of detection of 0.70 or greater. In certain embodiments, the methods have a sensitivity of detection of 0.80 or greater. In certain embodiments, the methods have a sensitivity of detection of 0.90 or greater. In certain embodiments, the methods have a sensitivity of detection of 0.95 or greater. In certain embodiments, the methods have a specificity of detection of 0.60 or greater. In certain embodiments, the methods have a specificity of detection of 0.70 or greater. In certain embodiments, the methods have a specificity of detection of 0.80 or greater. In certain embodiments, the methods have a specificity of detection of 0.90 or greater. In certain embodiments, the methods have a specificity of detection of 0.95 or greater.

Certain embodiments of the present invention provide methods of detecting a prostate cancer in a subject, the method comprising:
obtaining a biological sample from the subject;
processing the biological sample to allow detection of at least the following three endosomal markers: APPL1, SORT1, and SDC1;
detecting, in each biomarker, one or more of an altered presence, level, secretion and distribution of the selected biomarker in the processed sample; and
identifying a prostate cancer and/or the progression of the cancer in the subject.

Certain embodiments of the present invention provide methods for detecting a prostate cancer in a subject, the method comprising:
obtaining a biological sample from the subject;
processing the biological sample to allow detection of at least the following three endosomal markers: APPL1, SORT1, and SDC1;
comparing, in each biomarker, the presence, level, secretion and distribution of the selected markers; optionally with one or more other markers known to be indicative of the presence or absence of prostate cancer in the subject; and
identifying prostate cancer in the subject.

Certain embodiments of the present invention provide methods of detecting a prostate cancer in a subject, the method comprising:
processing a biological sample from said subject to allow detection of at least the following three endosomal markers: APPL1, SORT1, and SDC1;
comparing the presence, level, secretion and distribution of the selected markers; optionally with one or more other markers known to be indicative of the presence or absence of prostate cancer in the subject; and
identifying prostate cancer and/or the progression of the cancer in the subject.

Certain embodiments of the present invention provide methods of detecting a prostate cancer in a subject, the method comprising:
obtaining a biological sample from the subject;
processing the biological sample to allow detection of at least the following three endosomal markers: APPL1), SORT1, and SDC1; and
detecting one or more of an altered presence, level, secretion and distribution of these selected markers in the processed sample; and
identifying a prostate cancer and/or the progression of the cancer in the subject,
wherein the subject is effectively treated for the prostate cancer according to the biomarker pattern.

In one embodiment, the method comprises recommending a subject for active surveillance. The subject may be one who is found to have elevated levels of APPL1 and SORT1 and a decreased level of Syndecan-1 as compared to a reference.

In certain embodiments, the methods as described herein are used to diagnose prostate cancer in the subject, to screen for prostate cancer in the subject, for assessing prognosis, to determine the metastatic potential of a prostate cancer, to identify a subject suffering from prostate cancer, to identify a subject susceptible to prostate cancer, to determine the rate of relapse of prostate cancer in the subject, to determine the risk of mortality from prostate cancer in the subject, to stratify the prostate cancer, to discriminate between prostate cancer and not having prostate cancer in the subject, to determine whether the prostate cancer is an organ confined cancer, to discriminate between prostate cancer and one or more of benign prostatic hyperplasia, prostatitis and an inflammatory condition of the prostate, to determine pathogenic progression, to assess whether the prostate cancer is slow growing, indolent, or aggressive, to exclude the presence of prostate cancer in the subject, to identify a subject suitable for treatment and/or surgery for prostate cancer, and to determine the likelihood or risk of a subject having prostate cancer.

Certain embodiments of the present disclosure provide a method of detecting prostate cancer in a subject substantially as described herein with reference to any of the accompanying examples and/or figures.

Certain embodiments of the present disclosure provide a method or kit for identifying a subject suffering from, or susceptible to, a prostate cancer.

In certain embodiments, the method further comprises first identifying the level of PSA expression in the subject and stratifying the expression of the marker on the basis of the PSA expression level in the subject. PSA levels are as described herein.

In certain embodiments, the PSA is a level indicative of a low risk of a prostate cancer. In certain embodiments, the PSA level is less than 10 ng/mL.

Certain embodiments of the present invention provide methods of predicting the risk of recurrence of prostate cancer in a subject following a cancer therapy, the method comprising detecting one or more of an altered presence, level, secretion and distribution of at least the following three endosomal biomarkers APPL1, SORT1, and SDC1 in a biological sample of the subject as compared a reference.

Certain embodiments of the present invention provide methods of predicting the timing of recurrence of prostate cancer in a subject following a cancer therapy, the method comprising detecting one or more of an altered presence, level, secretion and distribution of at least the following three endosomal biomarkers APPL1, SORT1, and SDC1 in a biological sample of the subject as compared a reference.

The risk of recurrence may be a low, intermediate or a high risk of recurrence.

A high level of APPL1 and Syndecan-1 with minimal or no Sortilin-1 may predict a risk of clinical recurrence within about 50 months. A high level of APPL1 and Syndecan-1 with small amounts of Sortilin-1 may predict a risk of clinical recurrence over about 120 months. A high level of APPL1, Syndecan-1 and Sortilin-1 may predict that a subject is unlikely to have a clinical recurrence.

Certain embodiments of the present disclosure provide a kit for performing a method as described herein. The kits may comprise one or more components, reagents, and/or instructions as described herein. The reagents may include one or more antibodies that is capable of binding to APPL1, SORT1 and SDC1.

In certain embodiments, the kit comprises one or more reagents and/or instructions for determining the presence, level, expression, secretion and distribution of a selected biomarker.

The kit may comprise an antibody or fragment thereof capable of binding an amino acid sequence of any one of SEQ ID NO:1, SEQ ID NO: 2 or SEQ ID NO: 3.

The kit may comprise an antibody or fragment thereof capable of binding an amino acid sequence of SEQ ID NO: 4.

The kit may comprise an antibody or fragment thereof capable of binding an amino acid sequence of SEQ ID NO: 6 or 8.

Certain embodiments of the present invention also provide a composition comprising one or more antibodies or fragment thereof that binds to at least a group of the three endosomal biomarkers APPL1, SORT1, and SDC1 and a biological sample obtained from a subject having prostate cancer. The one or more antibodies or fragments thereof may be bound to a detectable label. Also provided herein is a method of preparing such a composition.

Certain embodiments of the present disclosure provide a method of treating a prostate cancer.

Certain embodiments of the present invention provide a method for diagnosing (or detecting) and treating a prostate cancer in a subject, the method comprising:
detecting APPL1, SORT1, and SDC1 from the subject; and
treating the subject based on one or more of the presence, level, secretion and distribution of the selected markers detected, with a cancer therapy.

Certain embodiments of the present invention provide a method for treating a prostate cancer in a subject, the method comprising:
detecting APPL1, SORT1, and SDC1 from the subject; and
treating the subject based on one or more of the presence, level, secretion and distribution of the selected markers detected, with a cancer therapy.

Certain embodiments of the present invention provide methods of determining the likelihood of the presence of a prostate cancer in a subject and treating the subject, the method comprising detecting one or more of an altered presence, level, secretion and distribution of at least the following three endosomal biomarkers APPL1, SORT1, and SDC1 in a biological sample from the subject as compared to a reference; and treating the subject found to have a likelihood of the presence of a prostate cancer.

Certain embodiments of the present invention provide methods of identifying a subject suffering from prostate cancer who is likely to be responsive to a cancer therapy, the method comprising detecting one or more of an altered presence, level, secretion and distribution of at least the following three endosomal biomarkers APPL1, SORT1, and SDC1 in a biological sample of the subject as compared a reference.

Also provided herein is a method of stratifying a subject suffering from prostate cancer into a likely responder or non-responder to a cancer therapy, the method comprising detecting elevated levels of at least a group of three endosomal biomarkers APPL1, SORT1, and SDC1 from a biological sample of the subject, when compared a reference.

The term “treating”, and related terms such as “treatment” and “treat”, refer to obtaining a desired effect in terms of improving the condition of the subject, ameliorating, arresting, suppressing, relieving and/or slowing the progression of one or more symptoms in the subject, a partial or complete stabilization of the subject, a regression of the one or more symptoms, or a cure of a disease, condition or state in the subject.

In certain embodiments, the treating comprising one or more of surgical intervention, radiation therapy and administration of a therapeutic agent.

A “cancer therapy” as used herein may also refer to one or more of surgical intervention, radiation therapy and administration of a therapeutic agent to treat a subject. Suitable therapies may also include the use of androgen-deprivation therapy (ADT) (such as leuprolide goserelin, triptorelin, histrelin, degerelix or surgical castration) or androgen receptor (AR) antagonists (such as MDV3100, ARN-509, flutamide, bicalutamide, nilutamide, or cyproterone acetate) or chemotherapeutics. Certain chemotherapeutics are well known for use against prostate cancer. These include capecitabine, carboplatin, cyclophosphamide (Cytoxan), cabazitaxel, daunorubicin, docetaxel (Taxotere), doxorubicin (Adriamycin), epirubicin (Ellence), fluorouracil (also called 5-fluorouracil or 5-FU), gemcitabine, eribulin, ixabepilone, methotrexate, mitomycin C, mitoxantrone, paclitaxel (Taxol), thiotepa, vincristine, vinorelbine.

In certain embodiments, the methods of the present invention relate to use in companion diagnostics for assessing the suitability of AR therapeutic intervention, non-AR therapy selection, AR therapeutic monitoring and PET scan and radiation therapy.

Certain embodiments of the present disclosure provide a method of treating a prostate cancer by surgical intervention to a subject based on one or more of the presence, level, expression, secretion and distribution of the selected marker detected, as described herein. Methods of surgical intervention for prostate cancer are known in the art.

Certain embodiments of the present disclosure provide a method of treating a prostate cancer by administering to a subject an effective amount of a therapeutic agent based on one or more of the presence, level, expression, secretion and distribution of the selected marker detected, as described herein. Methods of pharmacological intervention for prostate cancer are known in the art.

Certain embodiments of the present disclosure provide a method of treating a prostate cancer by radiation therapy based on one or more of the presence, level, expression, secretion and distribution of the selected marker detected, as described herein. Methods of radiation therapy for prostate cancer are known in the art.

In certain embodiments, the treatment occurs when one or more of the presence, level, expression, secretion and distribution presence of the selected marker is indicative of the presence of prostate cancer and/or an increased likelihood or risk of prostate cancer, as described herein.

In certain embodiments, one or more of an altered presence, level, expression secretion and distribution level of the selected biomarker is indicative that the subject is suitable for treatment. Alterations in the presence, level, expression, secretion, and distribution are as described herein.

In certain embodiments, an increased level of the selected biomarker is indicative that the subject is suitable for treatment. In certain embodiments, a decreased level of the selected marker is indicative that the subject is suitable for treatment. In certain embodiments, a down regulation of selected biomarker is indicative that the subject is suitable for treatment. In certain embodiments, an up regulation of the selected biomarker is indicative that the subject is suitable for treatment. In certain embodiments, a down regulation of one selected biomarker and/or an up-regulation of another selected biomarker is indicative that the subject is suitable for treatment.

Certain embodiments of the present disclosure provide a method of screening for a prostate cancer in a subject, the method comprising detecting at least the following three endosomal markers; APPL1, SORT1, and SDC1 from a subject sample.

Certain embodiments of the present invention provide a method of screening for a prostate cancer, and determining the progression thereof, in a subject, the method comprising detecting at least the following three endosomal markers: APPL1, SORT1, and SDC1 from a subject sample, wherein one or more of an altered presence, level, secretion and distribution of the selected marker is indicative of prostate cancer and/or progression thereof in the subject.

As described herein, certain embodiments of the present disclosure provide methods as follows: to diagnose prostate cancer in the subject, to screen for prostate cancer in the subject, for assessing prognosis, to determine the metastatic potential of a prostate cancer, to identify a subject suffering from prostate cancer, to identify a subject susceptible to prostate cancer, to determine the rate of relapse of prostate cancer in the subject, to determine the risk of mortality from prostate cancer in the subject, to stratify the prostate cancer, to discriminate between prostate cancer and not having prostate cancer in the subject, to determine whether the prostate cancer is an organ confined cancer, to discriminate between prostate cancer and one or more of benign prostatic hyperplasia, prostatitis and an inflammatory condition of the prostate, to determine pathogenic progression, to assess whether the prostate cancer is slow growing, indolent, or aggressive, to exclude the presence of prostate cancer in the subject, to identify a subject suitable for treatment and/or surgery for prostate cancer, and to determine the likelihood or risk of a subject having prostate cancer.

Certain embodiments of the present disclosure provide a method or kit to discriminate between a prostate cancer and one or more of benign prostatic hyperplasia, prostatitis and an inflammatory condition of the prostate in a subject.

Certain embodiments of the present disclosure provide a method or kit to determine pathogenic progression of a prostate cancer in a subject.

Certain embodiments of the present disclosure provide a method or kit to assess whether a prostate cancer in a subject is slow growing, indolent, or aggressive.

Certain embodiments of the present disclosure provide a method or kit to exclude the presence of a prostate cancer in a subject.

Certain embodiments of the present disclosure provide a method or kit to identify a subject suitable for treatment and/or surgery for prostate cancer.

Certain embodiments of the present disclosure provide a method or kit to determine the likelihood or risk of a subject having a prostate cancer.

Certain embodiments of the present disclosure provide a method or kit for identifying a selected marker for diagnosis and/or prognosis of a prostate cancer. Certain embodiments of the present disclosure provide a method of screening for a selected marker for diagnosis and/or prognosis of a prostate cancer.

Certain embodiments of the present disclosure provide isolated and/or purified antibodies, and/or antigen binding fragments thereof Antibodies and fragments thereof are as described herein. Antibodies, and antigen binding fragments thereof, may be used for example to detect a prostate cancer, such as for use in kits as described herein.

The term “antibody” is to be understood to mean an immunoglobulin molecule with the ability to bind an antigenic region of another molecule, and includes monoclonal antibodies, polyclonal antibodies, multivalent antibodies, chimeric antibodies, multispecific antibodies, diabodies and fragments of an immunoglobulin molecule or combinations thereof that have the ability to bind to the antigenic region of another molecule with the desired affinity including a Fab, Fab′, F(ab′)2, Fv, a single-chain antibody (scFv) or a polypeptide that contains at least a portion of an immunoglobulin (or a variant of an immunoglobulin) that is sufficient to confer specific antigen binding, such as a molecule including one or more Complementarity Determining Regions (CDRs).

In certain embodiments, the antibody (or antigen binding fragment thereof) comprises an affinity of at least 10⁶M⁻¹, at least 10⁷M⁻¹, at least 10⁸M⁻¹, at least 10⁹M⁻¹, at least 10¹⁰M⁻¹, at least 10¹¹M⁻¹, or at least 10¹²M⁻¹ to the antigen.

Antibodies may be generated using known methods in the art. For the production of antibodies, various hosts including goats, rabbits, rats, mice, humans, and others, may be immunized by injection with an appropriate antigen. Depending on the host species, various adjuvants may be used to increase an immunological response. Such standard adjuvants include Freund's adjuvant, mineral gels such as aluminium hydroxide, and surface-active substances such as lysolecithin, pluronic polyols, polyanions, peptides, oil emulsions, keyhole limpet hemocyanin, and dinitrophenol.

In certain embodiments, the antibody is a polyclonal antibody. Methods for producing and isolating polyclonal antibodies are known. In general, polyclonal antibodies are produced from B-lymphocytes. Typically polyclonal antibodies are obtained directly from an immunized subject, such as an immunized animal. Methods of immunization are known in the art.

In certain embodiments, the antibody is a monoclonal antibody. Monoclonal antibodies may be prepared using a technique that provides for the production of antibody molecules by continuous isolated cells in culture. These include, but are not limited to, the hybridoma technique, the human B-cell hybridoma technique, and the EBV-hybridoma technique. Methods for the preparation of monoclonal antibodies include for example Kohler et al. (1975) Nature 256:495-497 (herein incorporated by reference); Kozbor et al. (1985) J. Immunol. Methods 81:31-42 (herein incorporated by reference); Cote et al. (1983) Proc. Natl. Acad. Sci 80:2026-2030 (herein incorporated by reference); and Cole et al. (1984) Mol. Cell Biol. 62: 109-120 (herein incorporated by reference).

In certain embodiments, the antibody and/or an antigen binding fragment thereof comprises an isolated antibody. In certain embodiments, the antibody and/or an antigen binding fragment thereof comprise a purified antibody. Methods for producing and isolating polyclonal and monoclonal antibodies are known.

The term “isolated” refers to a species, such as a nucleic acid, a polypeptide or an antibody, that has been separated from its natural environment. Certain embodiments of the present disclosure provide an isolated nucleic acid, polypeptide, protein or antibody as described herein.

An isolated nucleic acid, polypeptide or antibody may be partially or substantially purified. In some cases, the isolated entity is in a substantially un-purified state, being associated with a variety of other species. In some cases, the isolated entity is in a substantially purified state, being substantially free of other substances with which it is associated in nature or in vivo. The term “purified” refers to a species that has undergone some form of process to increase the proportion of a desired species. Certain embodiments of the present disclosure provide a purified nucleic acid, polypeptide, protein or antibody as described herein.

In certain embodiments, the antibody has an isotype selected from the group consisting of IgG1, IgG2a, IgG2b, IgG3, IgM and IgA.

In certain embodiments, the antibody and/or an antigen binding fragment thereof is a mouse antibody and/or an antigen binding fragment thereof, a human antibody and/or an antigen binding fragment thereof, or a humanized antibody and/or an antigen binding fragment thereof. Other types of antibodies (or antigen binding fragments thereof) are contemplated.

Humanized antibodies, or antibodies adapted for non-rejection by other mammals, may be produced by a suitable method known in the art, including for example resurfacing or CDR grafting. In resurfacing technology, molecular modeling, statistical analysis and mutagenesis are combined to adjust the non-CDR surfaces of variable regions to resemble the surfaces of known antibodies of the target host. Strategies and methods for the resurfacing of antibodies, and other methods for reducing immunogenicity of antibodies within a different host are known, for example as described in U.S. Pat. No. 5,639,641. Humanized forms of the antibodies may also be made by CDR grafting, by substituting the complementarity determining regions of, for example, a mouse antibody, into a human framework domain.

Methods for humanizing antibodies are known. For example, the antibody may be generated as described in U.S. Pat. No. 6,180,370 (herein incorporated by reference); WO 92/22653 (herein incorporated by reference); Wright et al. (1992) Critical Rev. in Immunol. 12(3,4): 125-168 (herein incorporated by reference); and Gu et al. (1997) Thrombosis and Hematocyst 77(4):755-759) (herein incorporated by reference).

Humanized antibodies typically have constant regions and variable regions other than the complementarity determining regions (CDRs) derived substantially or exclusively from a human antibody and CDRs derived substantially or exclusively from the non- human antibody of interest.

Techniques developed for the production of “chimeric antibodies”, for example the splicing of mouse antibody genes to human antibody genes to obtain a molecule with appropriate antigen specificity and biological activity, may be performed by a suitable method. For example, chimeric antibodies may be produced as described in Morrison, S. L. et al. (1984) Proc. Natl. Acad. Sci 81:6851-6855 (herein incorporated by reference); Neuberger, M. S. et al. (1984) Nature 312:604-608 (herein incorporated by reference); and Takeda, S. et al. (1985) Nature 314:452-454 (herein incorporated by reference).

Immunoassays may be used for screening to identify antibodies and/or antigen binding fragments thereof having the desired specificity.

Antibody molecules and antigen binding fragments thereof may also be produced recombinantly by methods known in the art, for example by expression in E.coli expression systems. For example, a method for the production of recombinant antibodies is as described in U.S. Pat. No. 4,816,567 (herein incorporated by reference). Antigen binding fragments may also be produced, for example, by phage display technologies or using peptide libraries, which are known in the art.

Certain embodiments of the present disclosure provide an isolated or purified antibody, or an antigen binding fragment thereof, raised to a polypeptide as described herein. Certain embodiments of the present disclosure also provide polypeptides or proteins as described herein.

In certain embodiments, a polypeptide (or protein) as described herein is an isolated polypeptide. In certain embodiments, the polypeptide (or protein) as described herein is a purified polypeptide. In certain embodiments, a polypeptide (or protein) as described herein is a non-naturally occurring polypeptide. In certain embodiments, a polypeptide (or protein) as described herein is a recombinant polypeptide. In certain embodiments, a polypeptide (or protein) as described herein is a synthetic polypeptide. Other types of polypeptides are contemplated.

The term “variant” of a protein, polypeptide or of an amino acid sequence includes, for example, one or more of amino acid insertion variants, amino acid deletion variants, amino acid substitution variants, and amino acid modification variants (natural and/or synthetic).

For example, amino acid insertion variants may comprise amino- and/or carboxy-terminal fusions (of any desired length) and also insertions of single or two or more amino acids in a particular amino acid sequence. In the case of amino acid sequence variants having an insertion, one or more amino acid residues may be inserted into a particular site in an amino acid sequence, although random insertion with appropriate screening of the resulting product is also possible.

Amino acid deletion variants are characterized by the removal of one or more amino acids from the sequence. Amino acid substitution variants are characterized by at least one residue in the sequence being removed and another residue being inserted in its place.

Amino acid changes in variants may be non-conservative and/or conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

The polypeptides and amino acid variants described herein may be readily prepared with the aid of known peptide synthesis techniques such as, for example, by solid phase synthesis and similar methods or by recombinant DNA manipulation. The manipulation of DNA sequences for preparing proteins and peptides having substitutions, insertions or deletions, is described in detail in Sambrook, J, Fritsch, E. F. and Maniatis, T. Molecular Cloning: A Laboratory Manual 2nd. ed. Cold Spring Harbor Laboratory Press, New York. (1989), herein incorporated by reference, and Ausubel et al., Current Protocols in Molecular Biology (2011), John Wiley & Sons, Inc., herein incorporated by reference.

The term “derivatives” refers to a modified form of a species. For example, a derivative of a polypeptide or protein refers to a modified form of a polypeptide or protein. Such modifications include chemical modifications and comprise single or multiple substitutions, deletions and/or additions of any molecules associated with the protein or peptide, such as carbohydrates, lipids and/or proteins or peptides. The term “derivative” also extends to all functional chemical equivalents of said proteins and peptides.

Methods for isolating and/or producing polypeptides and protein are known, and are as described generally in Sambrook, J, Fritsch, E. F. and Maniatis, T. Molecular Cloning: A Laboratory Manual 2nd. ed. Cold Spring Harbor Laboratory Press, New York. (1989), herein incorporated by reference, and Ausubel et al., Current Protocols in Molecular Biology (2011), John Wiley & Sons, Inc., herein incorporated by reference.

Certain embodiments of the present disclosure provide a method of detecting an APPL1 protein or a fragment thereof, the method comprising using an antibody as described herein.

Certain embodiments of the present disclosure provide a kit comprising an antibody as described herein. The kit may comprise one or more other reagents as described herein.

Certain embodiments of the present disclosure provide a hybridoma producing an antibody as described herein. Methods for producing hybridomas and monoclonal antibodies are known in the art.

A typical protocol for the production of a hybridoma is as follows: Animals (e.g. mice) are first exposed to the selected antigen. Usually this is done by a series of injections of the antigen, over the course of several weeks. Once splenocytes are isolated from the mammal's spleen, the B cells may be fused with immortalised myeloma cells. The myeloma cells are generally selected to ensure they are not secreting antibody themselves and that they lack the hypoxanthine-guanine phosphoribosyltransferase (HGPRT) gene, making them sensitive to HAT medium. The fusion may be accomplished, for example, using polyethylene glycol or Sendai virus.

Fused cells are incubated in HAT medium for roughly 10 to 14 days. Aminopterin blocks the pathway that allows for nucleotide synthesis and unfused myeloma cells die, as they cannot produce nucleotides by the de novo or salvage pathways, because they lack HGPRT. Removal of the unfused myeloma cells is necessary because they have the potential to outgrow other cells, especially weakly established hybridomas. Unfused B cells die as they have a short life span. In this way, only the B cell-myeloma hybrids survive, since the HGPRT gene coming from the B cells is functional. These cells produce antibodies and are immortal. The incubated medium is then diluted into multi-well plates to such an extent that each well contains only one cell. Since the antibodies in a well are produced by the same B cell, they will be directed towards the same epitope, and are thus monoclonal antibodies.

The next stage is a rapid primary screening process, which identifies and selects only those hybridomas that produce antibodies of appropriate specificity. The hybridoma culture supernatant, secondary enzyme labeled conjugate, and chromogenic or fluorescent substrate, are then incubated, and the formation of a colored product indicates a positive hybridoma. Alternatively, immunocytochemical screening or flow cytometry can also be used.

The B cell that produces the desired antibodies can be cloned to produce many identical daughter clones. Supplemental media containing interleukin-6 are essential for this step. Once a hybridoma colony is established, it will continually grow in culture medium like RPMI-1640 (with antibiotics and fetal bovine serum) and produce antibodies.

Multiwell plates are used initially to grow the hybridomas, and after selection, are changed to larger tissue culture flasks. This maintains the well-being of the hybridomas and provides enough cells for cryopreservation and supernatant for subsequent investigations. The culture supernatant can yield 1 to 60 μg/ml of monoclonal antibody, which is maintained at −20° C. or lower until required.

By using culture supernatant or a purified immunoglobulin preparation, further analysis of a potential monoclonal antibody producing hybridomas can be made in terms of reactivity, specificity, and cross-reactivity.

Finally, standard techniques may be used for recombinant DNA technology, oligonucleotide synthesis, antibody production, peptide synthesis, tissue culture and transfection. Enzymatic reactions and purification techniques may be performed according to manufacturer's specifications or as commonly accomplished in the art or as described herein. The foregoing techniques and procedures may be generally performed according to conventional methods known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), herein incorporated by reference.

Exemplary embodiments are illustrated by the following examples. It is to be understood that the following description is for the purpose of describing particular embodiments only and is not intended to be limiting with respect to the above description.

Certain embodiments of the present disclosure provide an isolated and/or purified antibody binding to an epitope in an amino acid sequence in the human APPL1 protein comprising one or more of

ASSRPNQASSEG (SEQ ID NO: 1), SQSEESDLGEGGKKR (SEQ ID NO: 2), VPDPDPTKFPVNRN (SEQ ID NO: 3), and/or an equivalent region of a homolog, ortholog or paralog of the protein.

Certain embodiments of the present disclosure provide an isolated and/or purified antibody binding to an epitope in an amino acid sequence in the human Sortilin-1 protein comprising one or more of

WVSKNFGGKWEEIHK (SEQ ID NO: 4), EKDYTIWLAHSTDPE (SEQ ID NO: 5), and/or an equivalent region of a homolog, ortholog or paralog of the protein.

Certain embodiments of the present disclosure provide an isolated and/or purified antibody binding to an epitope in an amino acid sequence in the human Syndecan-1 protein comprising one or more of

TPRPRETTQLPT (SEQ ID NO: 8), and/or an equivalent region of a homolog, ortholog or paralog of the protein.

Certain embodiments of the present disclosure provide a method of detecting an APPL1 protein or a fragment thereof, the method comprising using an APPL1 antibody as described herein.

Certain embodiments of the present disclosure provide a method of detecting a Sortilin-1 protein or a fragment thereof, the method comprising using a Sortilin-1 antibody as described herein.

Certain embodiments of the present disclosure provide a method of detecting a Syndecan-1 protein or a fragment thereof, the method comprising using a Syndecan-1 antibody as described herein.

Certain embodiments of the present disclosure provide a method of detecting a prostate cancer in a subject, the method comprising using an anti-APPL1 antibody and/or an anti-Sortilin-1 antibody and/or an anti-Syndecan-1 antibody as described herein.

Other embodiments are disclosed herein.

In certain embodiments, the antibody, or antigen binding fragment thereof, is raised to one or more polypeptides consisting of an amino acid sequence of ASSRPNQASSEG (SEQ ID NO: 1), WVSKNFGGKWEEIHK (SEQ ID NO: 4), and EPKQANGGAYQKPTK (SEQ ID NO: 6), an antigenic fragment of any of the aforementioned amino acid sequences, and/or a variant of any of the aforementioned amino acid sequences or antigenic fragment thereof.

Certain embodiments of the present disclosure also provide polypeptides or proteins as described herein.

Certain embodiments of the present disclosure provide a polypeptide consisting of one or more of the following amino acid sequences: ASSRPNQASSEG (SEQ ID NO: 1), WVSKNFGGKWEEIHK (SEQ ID NO: 4), and EPKQANGGAYQKPTK (SEQ ID NO: 6), a fragment of any of the aforementioned amino sequences, an antigenic fragment of any of the aforementioned amino acid sequences, and/or a variant of any of the aforementioned amino acid sequences or an antigenic fragment thereof. In certain embodiments, the polypeptide is an isolated polypeptide. Such polypeptides may, for example, be used to raise an antibody.

Certain embodiments of the present disclosure provide a non-naturally occurring polypeptide comprising one or more of the following amino acid sequences: ASSRPNQASSEG (SEQ ID NO: 1), WVSKNFGGKWEEIHK (SEQ ID NO: 4), and EPKQANGGAYQKPTK (SEQ ID NO: 6), a fragment of any of the aforementioned amino sequences, an antigenic fragment of any of the aforementioned amino acid sequences, and/or a variant of any of the aforementioned amino acid sequences or an antigenic fragment thereof. In certain embodiments, the polypeptide is an isolated polypeptide. Such polypeptides may, for example, be used to raise an antibody.

Certain embodiments of the present disclosure provide an isolated and/or purified antibody binding to an epitope in an amino acid sequence in the human APPL1 protein comprising one or more of

ASSRPNQASSEG (SEQ ID NO: 1), SQSEESDLGEGGKKR (SEQ ID NO: 2), VPDPDPTKFPVNRN (SEQ ID NO: 3), and/or an equivalent region of a homolog, ortholog or paralog of the protein. Methods for identifying the equivalent binding regions of related targets are known in the art.

Certain embodiments of the present disclosure provide an isolated and/or purified antibody binding to an epitope in an amino acid sequence in the human Sortilin-1 protein comprising one or more of

WVSKNFGGKWEEIHK (SEQ ID NO: 4), EKDYTIWLAHSTDPE (SEQ ID NO: 5), and/or an equivalent region of a homolog, ortholog or paralog of the protein. Methods for identifying the equivalent binding regions of related targets are known in the art.

Certain embodiments of the present disclosure provide an isolated and/or purified antibody binding to an epitope in an amino acid sequence in the human Syndecan-1 protein comprising one or more of

TPRPRETTQLPT (SEQ ID NO: 8), and/or an equivalent region of a homolog, ortholog or paralog of the protein. Methods for identifying the equivalent binding regions of related targets are known in the art.

In certain embodiments, a polypeptide (or protein) as described herein is an isolated polypeptide. In certain embodiments, the polypeptide (or protein) as described herein is a purified polypeptide. In certain embodiments, a polypeptide (or protein) as described herein is a non-naturally occurring polypeptide. In certain embodiments, a polypeptide (or protein) as described herein is a recombinant polypeptide. In certain embodiments, a polypeptide (or protein) as described herein is a synthetic polypeptide. Other types of polypeptides are contemplated.

The term “variant” of a polypeptide or of an amino acid sequence includes, for example, one or more of amino acid insertion variants, amino acid deletion variants, amino acid substitution variants, and amino acid modification variants (natural and/or synthetic). For example, amino acid insertion variants may comprise amino- and/or carboxy-terminal fusions (of any desired length) and also insertions of single or two or more amino acids in a particular amino acid sequence. In the case of amino acid sequence variants having an insertion, one or more amino acid residues may be inserted into a particular site in an amino acid sequence, although random insertion with appropriate screening of the resulting product is also possible.

Amino acid deletion variants are characterized by the removal of one or more amino acids from the sequence. Amino acid substitution variants are characterized by at least one residue in the sequence being removed and another residue being inserted in its place.

### EXAMPLES

### Example 1—Interpretation of Tissue Immunochemistry With Conventional Light Microscopy

### (i) Antibody Reagents

Anti-APPL1 mouse monoclonal antibodies were generated (Genscript, Piscataway, NJ 08854, USA.) using the peptide sequence ASSRPNQASSEG (SEQ ID NO: 1), SQSEESDLGEGGKKR (SEQ ID NO: 2), and VPDPDPTKFPVNRN (SEQ ID NO: 3).

Anti-Sortilin-1 mouse monoclonal antibodies were generated (Genscript, Piscataway, NJ 08854, USA.) using the peptide sequence WVSKNFGGKWEEIHK (SEQ ID NO: 4) and EKDYTIWLAHSTDPE (SEQ ID NO: 5).

Anti-Syndecan-1 mouse monoclonal antibodies were generated (Genscript, Piscataway, NJ 08854, USA.) using the peptide sequence EPKQANGGAYQKPTK (SEQ ID NO: 6), SHPHRDMQPGHHETS (SEQ ID NO: 7), and TPRPRETTQLPT (SEQ ID NO: 8),

### (ii) Methods

### Preparation of Histology Sample:

Block preparation—paraffin wax embedded blocks were sectioned at 2 μm, floated (Reverse osmosis purified water at 42° C.) onto coated Super frost Plus slides and air dried. Sections were then baked at 60° C. for 1 hour and stored at 4° C. Serial sections were cut for all analysis with the first stained with routine Haematoxylin and Eosin (outlined below) and the last section used for a negative control.

Sections were cut on the Microtom HM325 or the Leica Histo core AutoCut microtomes.

### (a) Hematoxylin and Eosin (H & E) Staining

### Reagents:

Ehrlich's Haematoxylin
Eosin solution
   95% ethanol
   1% aqueous eosin Y (E4009, Sigma-Aldrich)
   1% phloxine (P2759, Sigma-Aldrich)
   Glacial acetic acid (0.05% (V/V)
Acid Alcohol
   1% hydrochloric acid (30% stock) in 70% ethanol
Ammonia water
   0.04% aqueous ammonia

### Protocol:

H&E sections were stained using an automated ST5010 Leica Autostainer XL as outlined in the table below. Sections were then cover slipped using coverslips with a thickness number of 1.5 (170 μm).

| STEP | REAGENT | TIME | |
|---|---|---|---|
| 1 | Oven | 10 | minutes |
| 2 | Xylene | 2 | minutes |
| 3 | Xylene | 2 | minutes |
| 4 | 100% ethanol | 2 | minutes |
| 5 | 100% ethanol | 2 | minutes |
| 6 | 90% ethanol | 1 | minutes |
| 7 | Wash 1 | 2 | minutes |
| 8 | Hematoxylin | 25 | minutes |
| 9 | Wash 2 | 2 | minutes |
| 10 | 1% HCL in 70% Ethanol | 1 | second |
| 11 | Wash 3 | 3 | minutes |
| 12 | Ammonia water | 2 | minutes |
| 13 | Wash 4 | 2 | minutes |
| 14 | RO water | 1 | minute |
| 15 | Eosin | 4 | minutes |
| 16 | Wash 5 | 2 | minutes |
| 17 | 90% ethanol | 1 | minute |
| 18 | 100% ethanol | 2 | minutes |
| 19 | 100% ethanol | 2 | minutes |
| 20 | Xylene | 2 | minutes |
| 21 | Xylene | 2 | minutes |

| | | |
|---|---|---|
| 22 | Xylene | ∞ |
| 23 | coverslip | |

### (b) Immunohistochemistry Protocol.

Immerse sections in clean xylene for 5 minutes. Drain excess xylene and immerse sections in 100% ethanol. Agitate sections then check for remaining wax (white). Immerse sections for 1 minute; drain and place in 90% ethanol for an additional 1 minute. Immerse in running tap water for 2 minutes. If wax or ethanol is remaining a streaming artefact will be observed.

Antigen retrieval; HIER—Heat Induced Epitope Retrieval: Immerse sections in citrate buffer pH 6 or TRIS EDTA pH 9. Ensure the last section in the rack is facing the others to avoid damaging the section during heating. Include another 250 mL of water to act as a heat sink if only using one. Heat on high for 4 minutes until boiling and continue on medium low for 15 minutes. Remove entire container and place in a cool water bath for 30 minutes. Wash sections in 1× TBS for 5 minutes.

Quench endogenous peroxidase using freshly prepared 3% H₂O₂ in TBS for 5 minutes. Wash sections in 1×TBS for 2 minutes. Apply DAKO Pap pen around sections. Do not allow sections to dry out.

Apply primary antibody to the sections. Ensure the solution reaches all the way to the pap pen to prevent drying. Incubate sections in a moist humid chamber for 1 hour at room temperature. If necessary, cover with parafilm.

Wash sections 3× in 1×TBS for 2 minutes each.

Apply visualisation reagent to the sections. Ensure the solution reaches all the way to the pap pen to prevent drying. Incubate sections in a moist humid chamber for 30 minutes at room temperature. If necessary, cover with parafilm.

Wash sections 3× in 1×TBS for 2 minutes each.

Make DAB solution—1 drop of DAB reagent in 1 mL of substrate solution during last 2 minute wash. Apply this DAB solution for exactly 10 minutes to the sections at room temperature. Immediately wash sections with distilled H₂O. Continue washing with distilled H₂O for 2 minutes.

Counter stain nuclei in Ehrlich's Haematoxylin for 1 minute. After counter staining, immediately immerse sections in running RO H₂O until the purple runs clear.

Differentiate the Haematoxylin staining with 1% Acid in 70% Alcohol. Immerse sections in the acid alcohol for 1-2 seconds and immediately wash in running RO H₂O.

“Blue” in 0.04% Ammonia H₂O for 1 minute. And wash in water.

Dehydrate in a graded series of ethanol concentrations, clear in xylene and mount in PIX: Immerse sections in 90% ethanol for 10 seconds and agitate, transfer sections to 100% ethanol for a further 10 seconds. Clear sections for 15 seconds in xylene and mount using 24×50 mm cover slips and PIX mountant. Allow to dry either over night at Room temperature or on the solid state heat plate at 60° C.

### ii) Evaluation of the Biomarkers

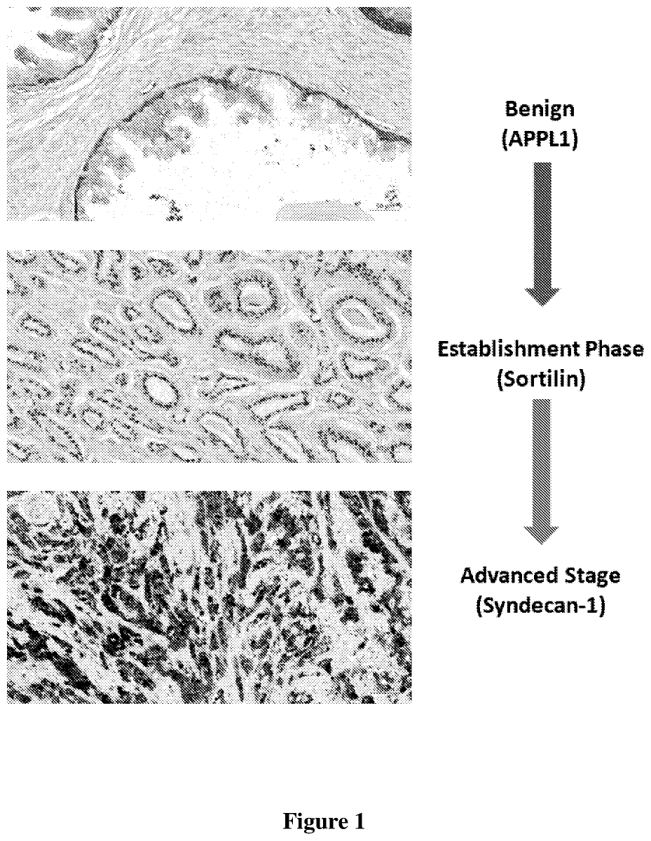

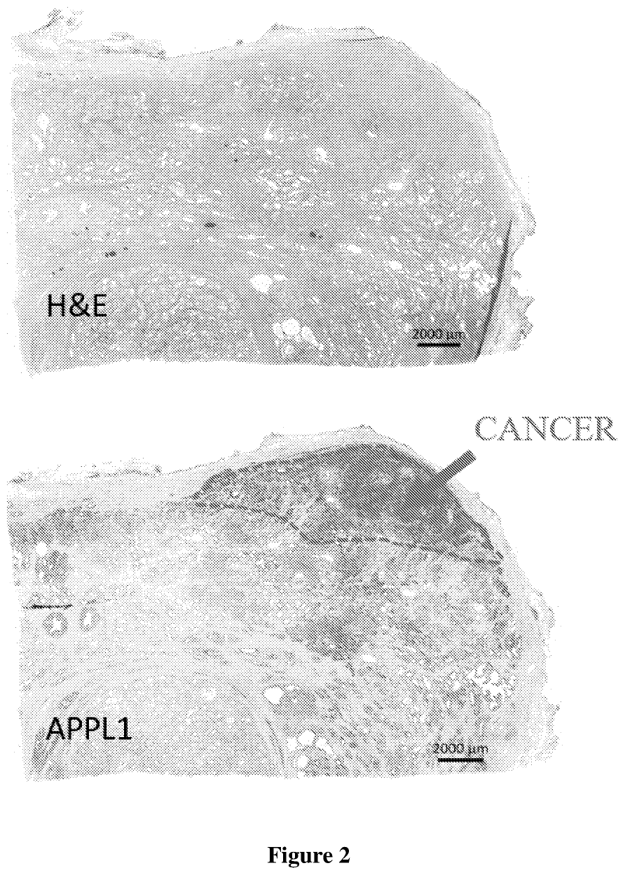

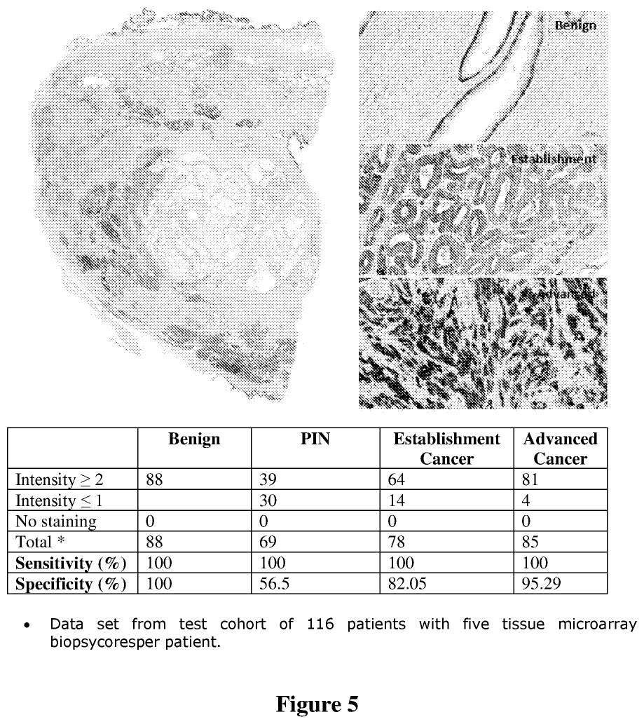

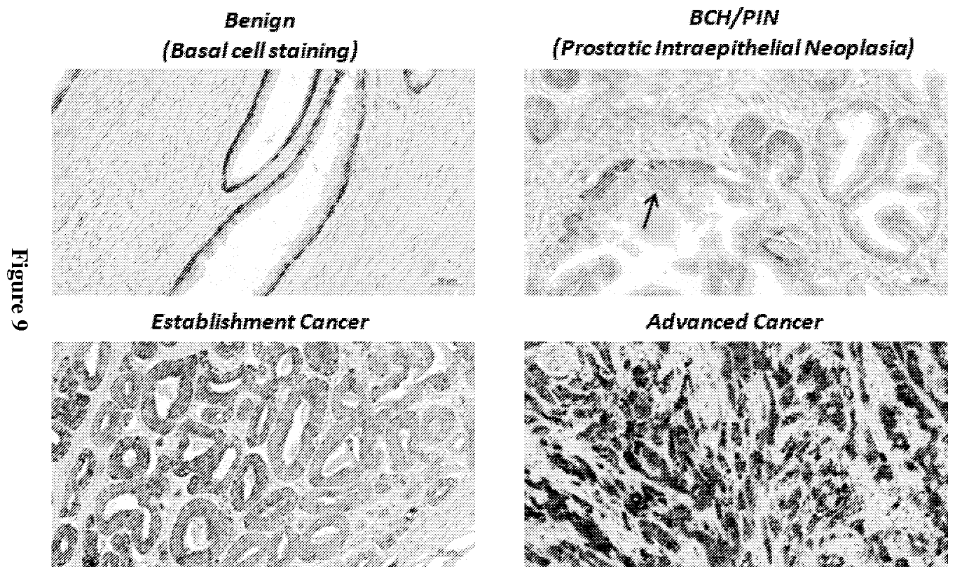

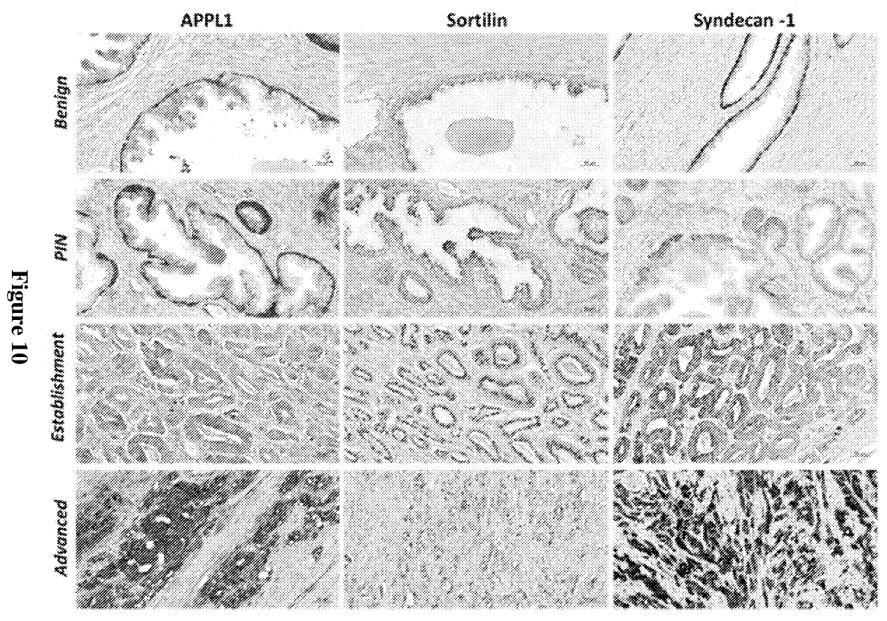

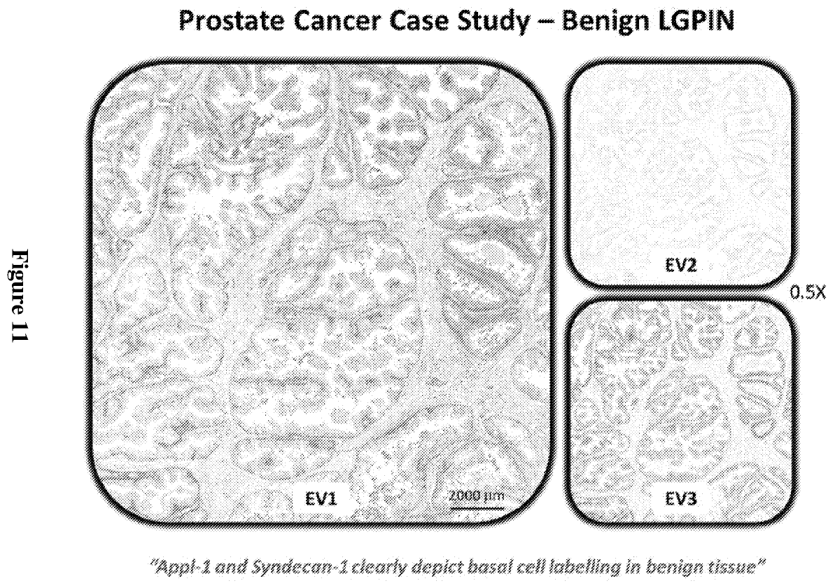

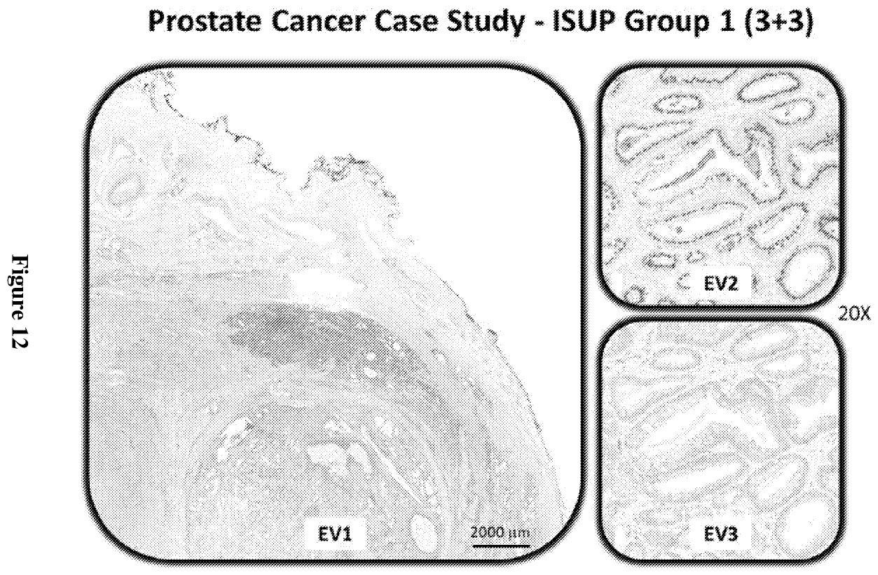

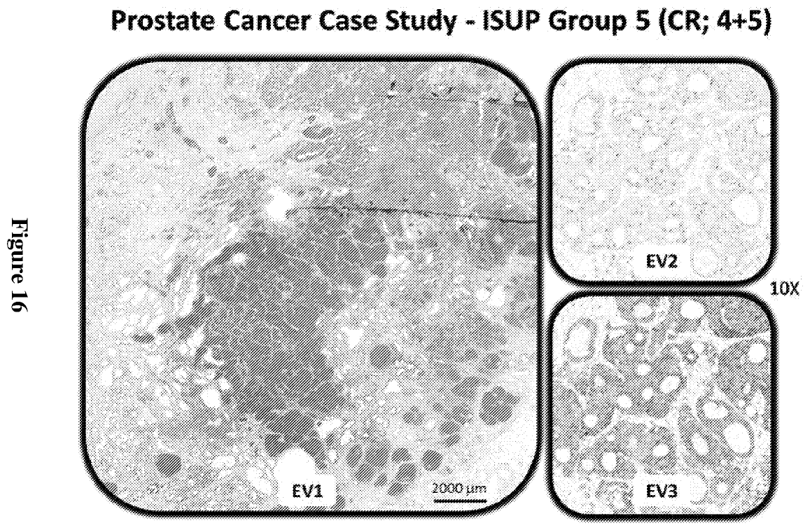

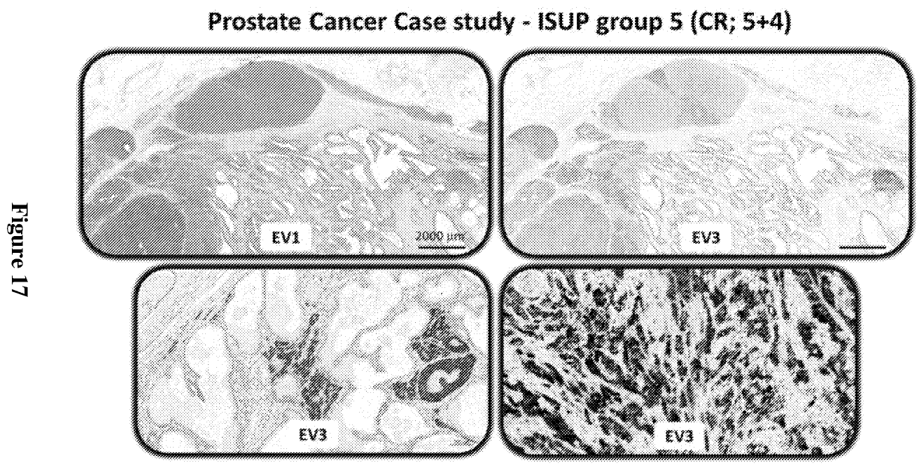

(a) First, evaluate the APPL1 stained section at low power (4×) magnification. This may highlight areas of cancer and its boundaries. Areas of cancer are obvious with APPL1 at low power and staining intensity may increase in areas of advanced cancer (FIGS. 2, 3, 7, 10 and larger panels of FIGS. 11-17).
(b) Next, utilise both Sortilin-1 and Syndecan-1 together to score areas of low-grade (Gleason Grade 3) and high-grade (Gleason Grade 4) cancer, respectively
   iii) Interpretation of Tissue Immunochemistry
(a) Sortilin-1 staining presents with strong staining in low-grade cancer (Gleason Grade 3) and sometimes may present with reduced staining in areas of high-grade cancer (>Gleason Grade 3; FIGS. 1, 4, 8 10 and FIGS. 12-16). In areas of low-grade cancer, Sortilin-1 staining is very polar and is distributed in a supranuclear position (FIGS. 1, 4, 8, 10, 12-14). Loss of Sortilin-1 staining intensity and loss of polarity of staining may sometimes indicate progression to more advanced cancer (>Gleason Grade 3).
(b) Low power magnification with Syndecan-1 detects benign glands with strong basal cell staining (FIG. 5, 9, 10) and absent or weak staining in the secretory epithelial cell layer and PIN tissue (FIG. 9, 10). Syndecan-1 staining presents with moderate to high staining intensity in areas of Gleason Grade 4 and Gleason Grade 5 cancer. Syndecan-1 can be detected in areas of establishment cancer as it advances towards Gleason Grade 4 (FIGS. 9, 10, 14). Areas of advanced cancer with migrating fronts may show strong characteristic staining of Syndecan-1, while also highlighting single cancer cells (FIGS. 9, 10, 16, 17). Strong Syndecan-1 staining is present in areas of high-grade advanced cancer (FIGS. 9, 10, 14, 15, 16, 17).

### Example 2—Diagnosis of Prostate Cancer and Treatment Options on the Basis of the Diagnostic/Prognostic Potential of the Biomarker(S)

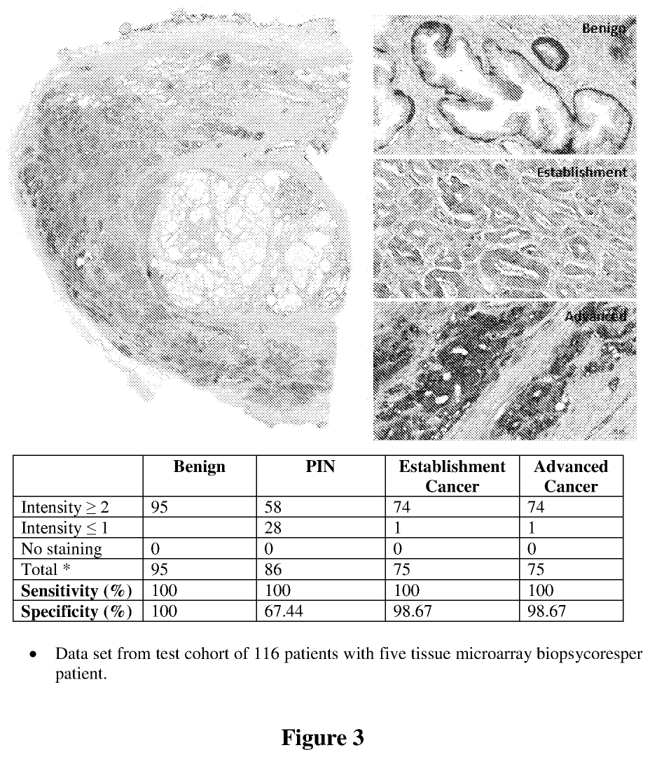

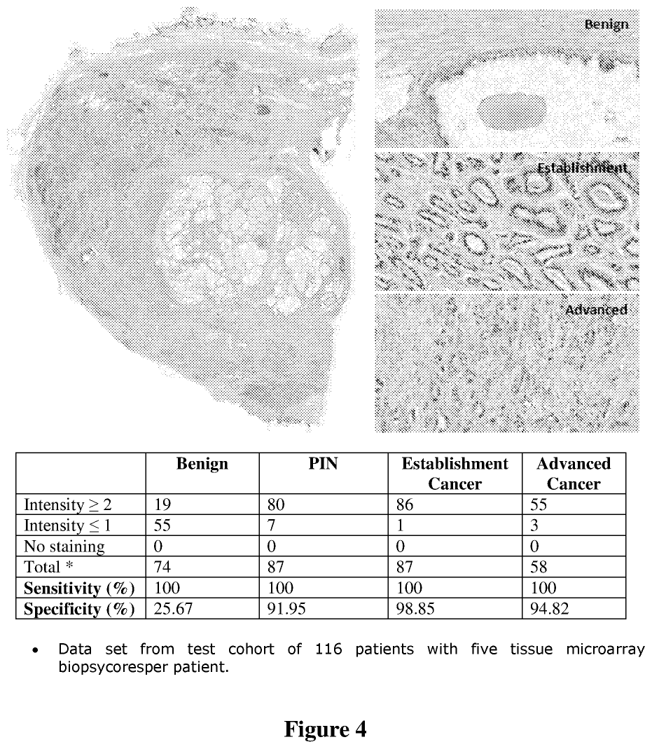

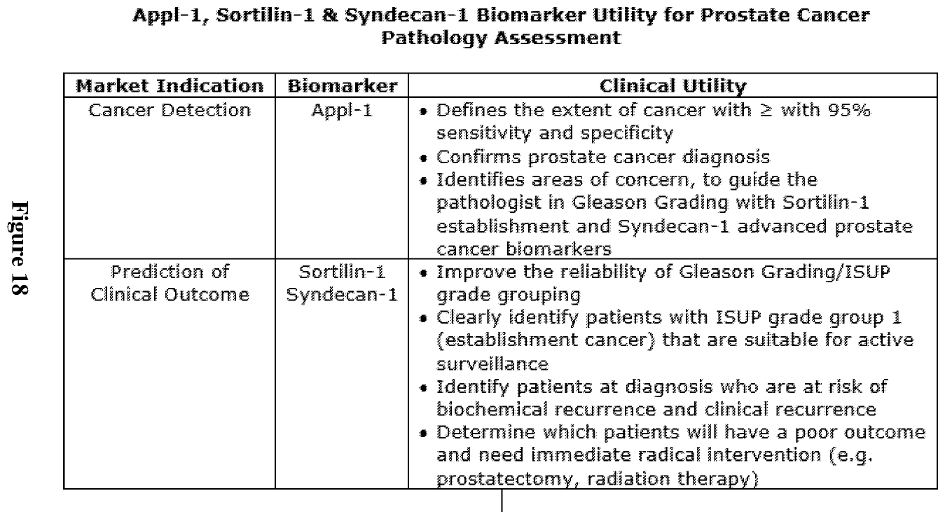

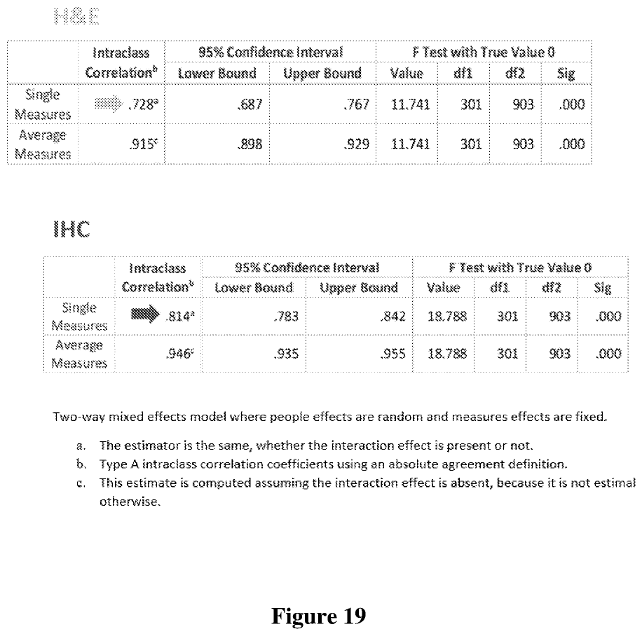

The three biomarkers APPL1, Sortilin-1 and Syndecan-1 map prostate cancer pathogenesis and the latter two identify two distinct metabolic stages of prostate cancer. The biomarker APPL1 can be used to accurately confirm the diagnosis of prostate cancer (≥95% sensitivity and specificity; for example see FIG. 3), while two additional biomarkers provide high specificity for the specific stages of cancer progression (see for example FIGS. 4 and 5 respectively) and can be used for prognosis (Summarised in FIG. 18 with examples of clinical practice uses in FIGS. 19-23). Sortilin-1 has a specific polarised distribution and increased expression in establishment phase cancer and can be used to identify patients that are suitable for active surveillance; where there is limited to no Syndecan-1 staining (See case study FIG. 12). Syndecan-1 detects advanced phase cancer and together APPL1, Sortilin-1 and Syndecan-1 can be used to align tissue pathology with patient outcomes (biochemical and clinical recurrence), providing a reliable method for prognosis (FIG. 12-18, 20-22).

### (i) Methods—as For Example 1

### (ii) APPL1 Provides Superior Cancer Visualisation

APPL1 maps the geography of the cancer in prostatectomy patient tissue see FIGS. 2, 3 and case studies FIGS. 12-17). APPL1 shows cytoplasmic distribution in cancer tissue and expression increases from establishment to advanced cancer there is higher intensity staining (e.g. FIG. 3), which also shows a cytoplasmic distribution (FIGS. 2, 3 & 7). The APPL1 biomarker is therefore able to accurately distinguish benign and cancer tissue, based on specific biomarker distribution. This enables very precise identification of cancer tissue (see case studies FIGS. 12-17), facilitating easy visualisation of the cancer geography and defining of the boundaries of the prostate cancer (FIGS. 2, 3). This biomarker effectively maps the cancer regions (e.g. FIG. 2; dashed line) and enables accurate confirmation of the diagnosis of prostate cancer in patient tissue samples (FIG. 3). In clinical practice this allows the pathologist to reliably confirm the presence of the cancer (FIG. 18, 19) and to see the extent of its spread (FIG. 2 and case studies FIGS. 12-17). Because the biomarker is connected to the pathogenic process it allows a complete pictorial view of the cancer. APPL1 intensely stains the basal cell layer in benign tissue (FIG. 3, 7, 10, 11) and has a quantitative increase in expression and a change in distribution to the cytoplasm of cells in establishment cancer tissue (e.g. FIG. 3). APPL1 intensely stains advanced cancer tissue (e.g. FIG. 3, 7, 10, 15-17). APPL1 displays high sensitivity and specificity and is almost unique as very few biomarkers achieve this performance for any cancer in clinical practice (table in FIG. 3 shows data for APPL1).

(iii) Sortilin-1 Defines Cancer Establishment and Tends to Have Lower Expression and a Different Distribution in Advanced Cancer

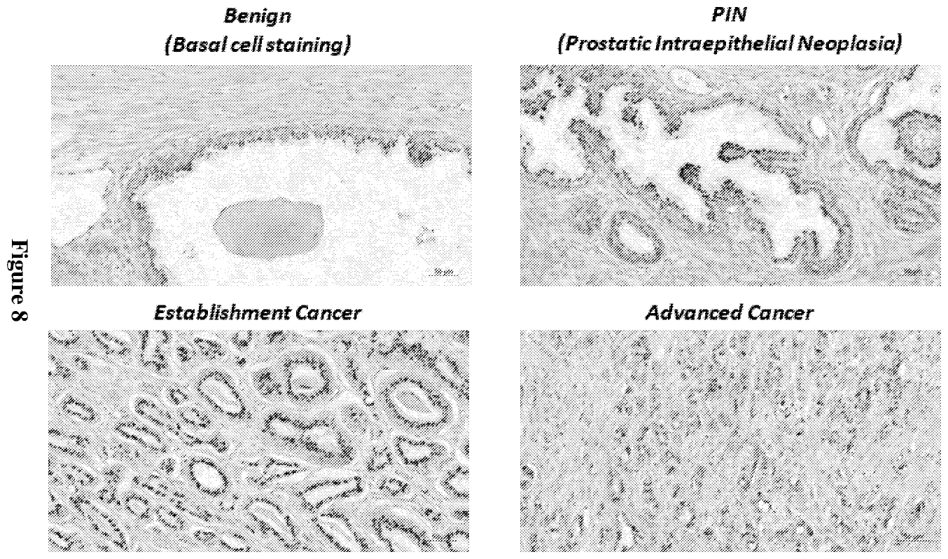

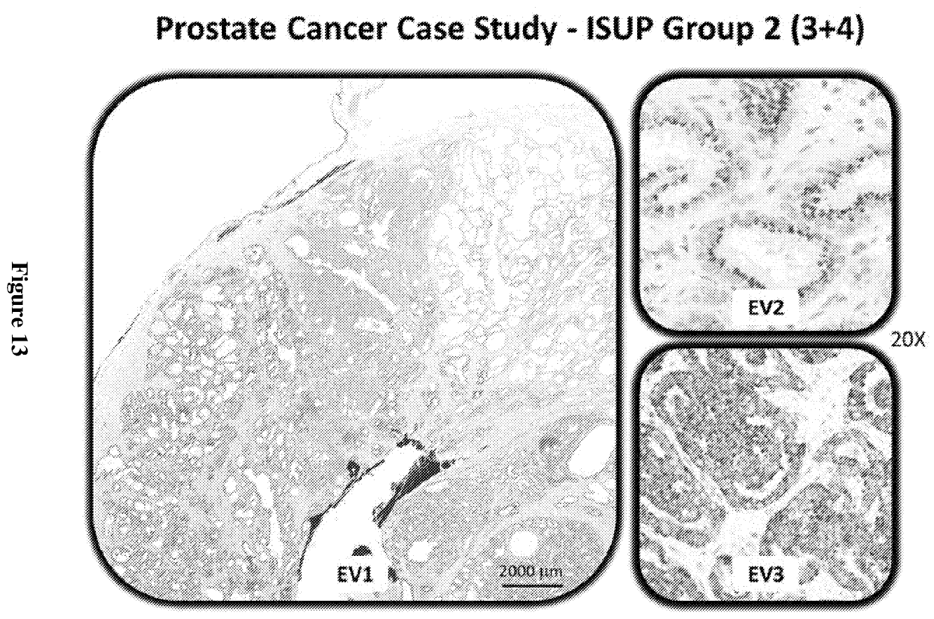

Sortilin-1 has minimal to no expression in benign tissue, except where cancer cells start to form PIN tissue (FIG. 8); and the biomarker then increases in expression and shows a polarized distribution in establishment cancer (FIGS. 1, 4, 8, 10 and case study FIG. 13). Sortilin-1 has a very characteristic polarized distribution in early or establishment cancer (e.g. FIGS. 4, 8) and all elements of this pathway are androgen regulated including the GLUT-4 transporter. Sortilin-1 controls the biogenesis of this critical pathway and specifically the GLUT-4/GLUT-1 vesicle formation and this effectively visualizes cancer cells using anaerobic glycolytic metabolism or “so called” Warburg metabolism. In advanced cancer Sortilin-1 shows reduced expression and the distribution is not polarized (e.g. FIGS. 4, 8). This change in biomarker expression is indicative of cancer tissue that is switching from glucose to lipid metabolism and Sortilin-1 is integrally involved in this switching process. Sortilin-1 is expressed uniformly in cancer tissue that has previously been shown to be Gleason grade 3 and is therefore ideal for identifying cancer patients that fall into ISUP grade group 1 and who are suitable for active surveillance (e.g. Case study FIG. 12).

### (iv) Syndecan-1 Defines Advanced Prostate Cancer.

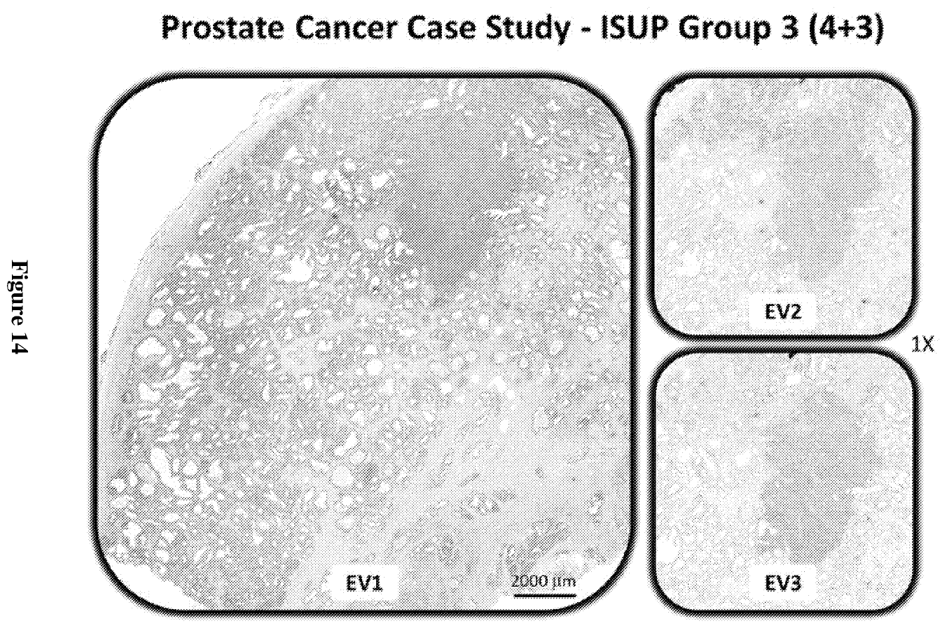

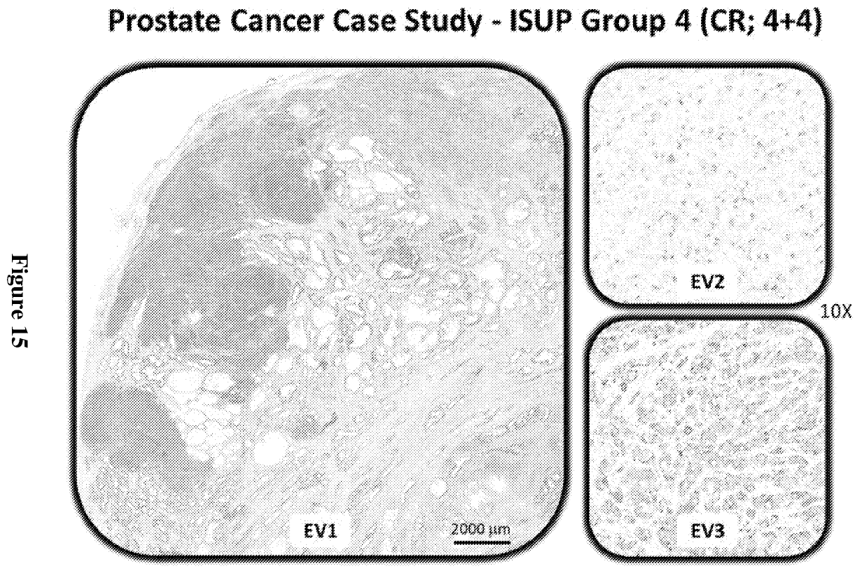

Syndecan-1 has very strong expression in basal cells, which is similar to APPL1, but is lost as PIN tissue is formed (FIGS. 5, 9, & 10). In early or establishment cancer the Syndecan-1 biomarker has limited expression except where cancer cells are changing to a more lipid base metabolism (e.g. FIG. 5, 9, and case study FIG. 14). While the switch in metabolism is controlled by reducing the amount of Sortilin-1 this releases enzymes and growth factors that stimulate lipid metabolism. Syndecan-1 signals this change and augments the growth factor signaling, stimulates the signaling cascade that effects cell migration, augments platelet binding and immune cloaking and participates directly in the process of advanced cancer. This enables Syndecan-1 to be used to specifically map advanced cancer, identifying multiple nodes and clearly depicting where the cancer has broken the capsule (e.g. FIGS. 5, and case study FIGS. 15-17). This biomarker can therefore accurately detects advanced cancer and the balance of the amount of Syndecan-1 and Sortilin-1 enables a more accurate Gleason grading/ISUP grade grouping in clinical practice (see FIGS. 18-22).

### (v) The Combination of the Biomarkers; APPL1, Sortilin-1 and Syndecan-1 Provide Reliable Prostate Cancer Assessment.

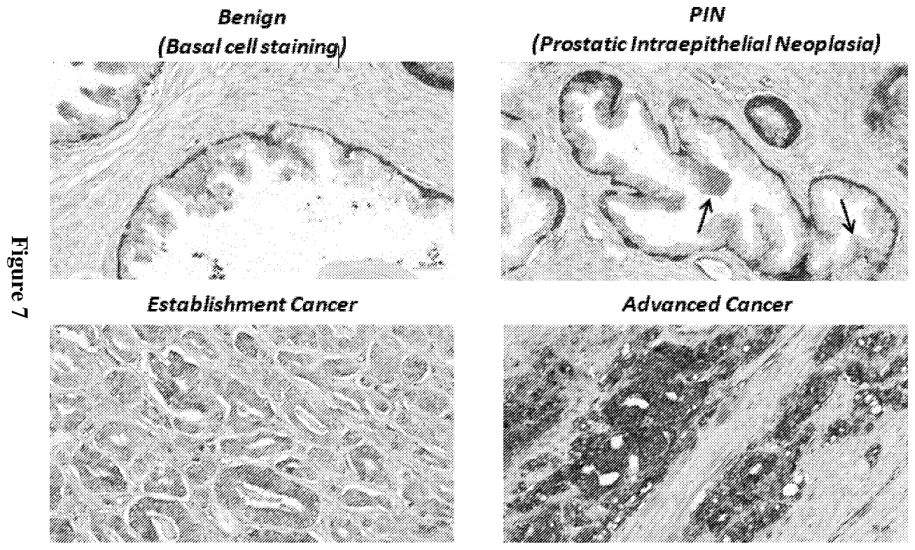

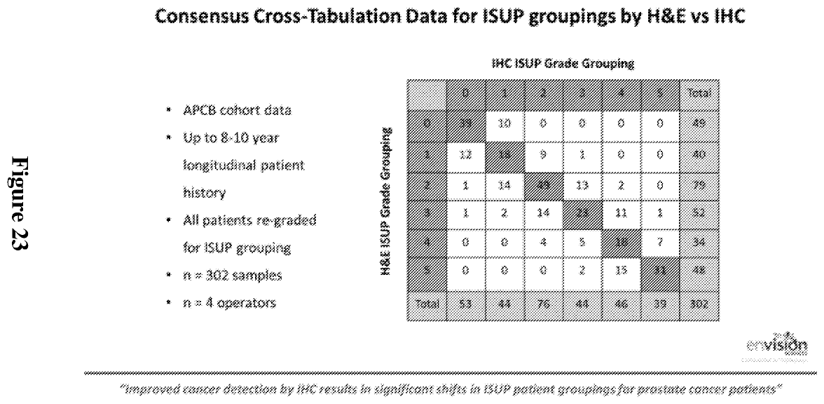

APPL1 defines the extent of the cancer with >95% sensitivity and specificity to accurately confirm a diagnosis of prostate cancer on patient tissue samples (see FIGS. 3, 18, 19). In clinical practice the APPL1 biomarker is used to map regions of interest for specific grading with Sortilin-1 and Syndecan-1 (see case studies FIGS. 12-17), but the increased expression of APPL1 also gives a clear indication of cancer progression (e.g. FIGS. 3, 7, and case studies FIGS. 15-17). The reliability of Gleason grading using H&E histology staining is only moderate with a Kappa value of <0.600, while ISUP grade grouping increases the Kappa value to 0.728 which is substantial, but by using Sortilin-1 and Syndecan-1 IHC the reliability of ISUP grade grouping increases significantly to a Kappa value of 0.814; which is in the almost perfect range (FIG. 19). With this increased reliability of cancer detection and grading there are very significant changes in patient grade groupings (FIG. 23). This new technology will provide unprecedented advice to clinicians and patients for life-saving therapeutic intervention by providing accurate diagnosis and prognosis, improving selection for active surveillance and for the first-time identifying patients with high risk of metastasis who need immediate intervention.

### (vi) The Combination of the Biomarkers; APPL1, Sortilin-1 and Syndecan-1 Can Assist in Assigning ISUP Grade Grouping

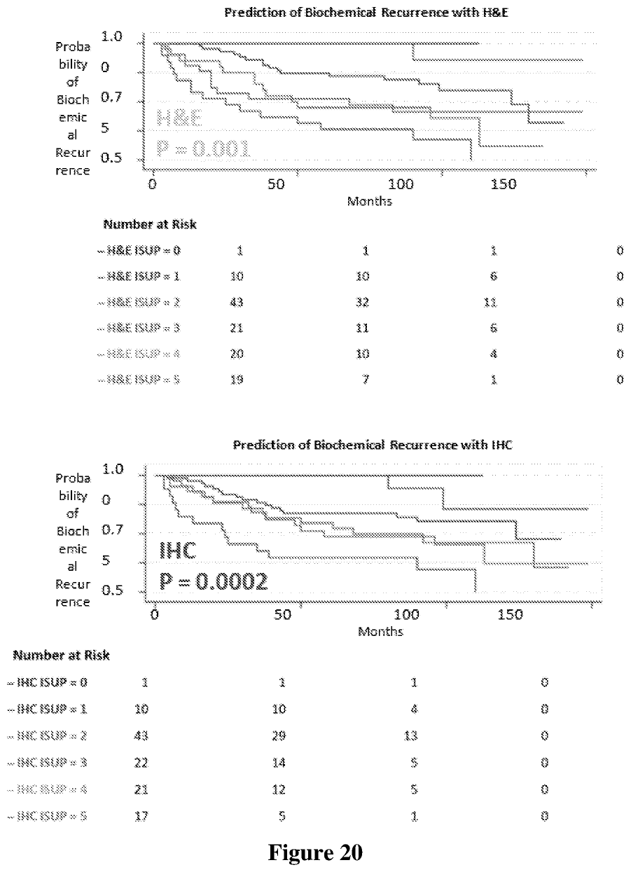

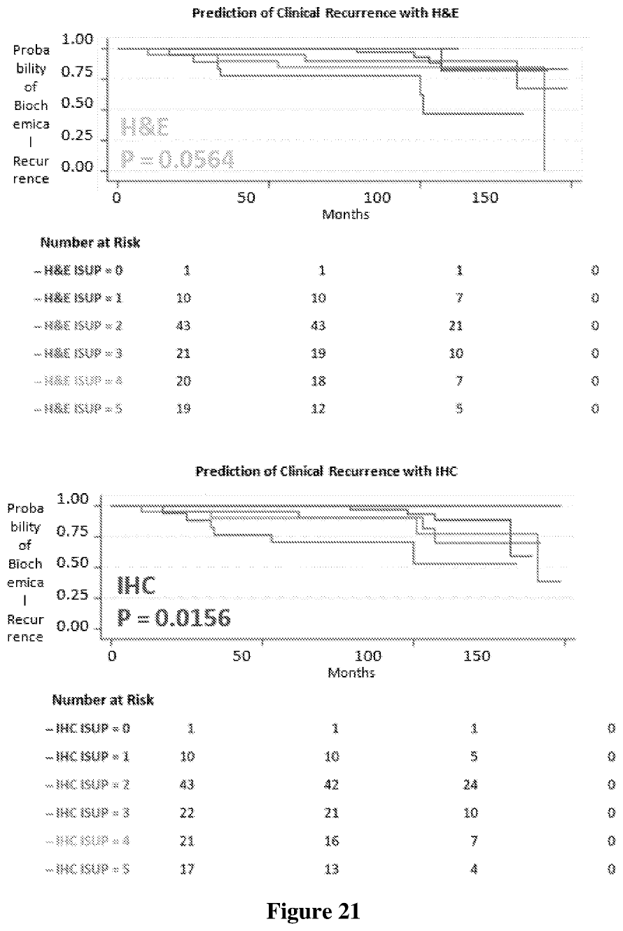

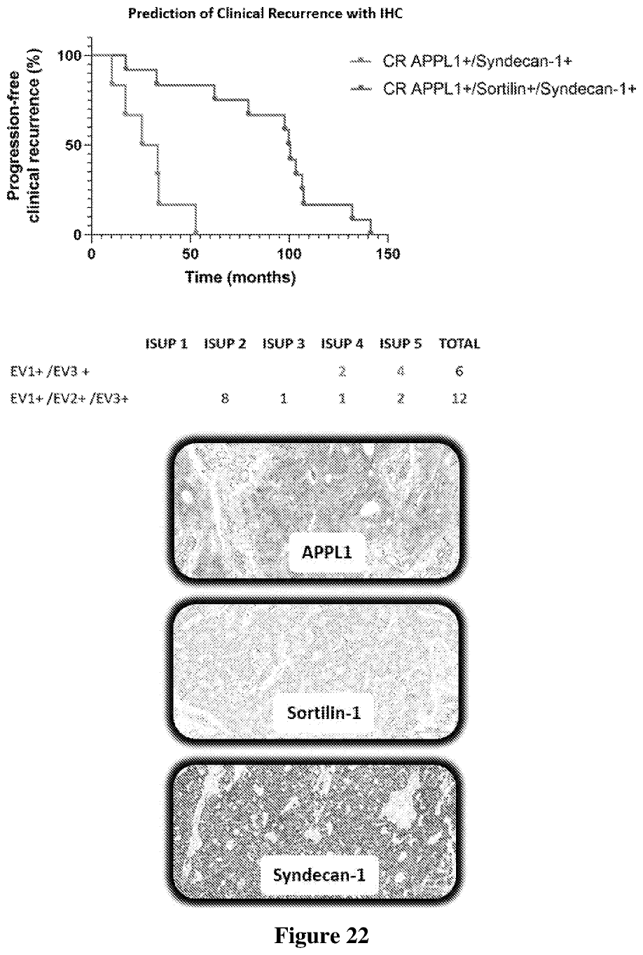

ISUP grade grouping using H&E has a significant capacity to predict biochemical recurrence (BCR; P=0.001) but the biomarkers Sortilin-1 and Syndecan-1 significantly increase this capacity to detect BCR (P=0.0002) (FIG. 20). More importantly, while H&E based ISUP grade grouping cannot predict clinical recurrence (CR), by using IHC with the Sortilin-1 and Syndecan-1 biomarkers to do the ISUP grading this results in a significant capacity to predict CR (FIG. 21). The combination of APPL1, Sortilin-1 and Syndecan-1 also displays specific patterns of biomarker expression, and when used in combination the biomarkers are able to predict the timing of clinical recurrence (FIG. 22). A high level of APPL1 with minimal or no Sortilin-1 and high level of Syndecan-1 biomarker staining depicts patients that go to clinical recurrence within ˜50 months, while patients that have high APPL1 and Syndecan-1 with small amounts of Sortilin-1 go to clinical recurrence over —120 months (FIGS. 22). Patients that have high levels of Sortilin-1 do not go to clinical recurrence. This technology provides the first ever prediction of clinical recurrence which is directly linked to metastasis and survival and has profound implications for patient management.

### Example 3—Syndecan-1 Peptide; EPKQANGGAYQKPTK (SEQ ID NO. 6) is the Optimum Peptide for Antibody Production to Syndecan-1 on the Basis of the Optimal Detection of Advanced Cancer in Patient Tissue

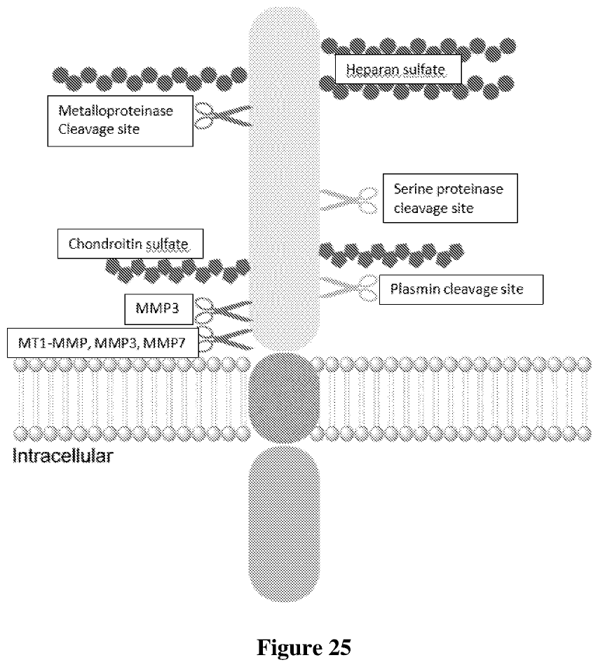

Syndecan-1 (SDC1) is a transmembrane proteoglycan that contains both heparan sulphate and chondroitin sulphate chains. Syndecan-1 is composed of a 310 amino acids long core protein, which consists of an extracellular domain (ectodomain), a transmembrane domain and a cytoplasmic domain (FIG. 25). It plays important roles in regulating a number of important processes, including growth factor uptake, cell adhesion, cell migration, endocytosis, exosorne biogenesis, and fibrosis. Syndecan-1 can be found in two forms: membrane-incorporated and soluble. The soluble form is the ectodomain containing proteoglycan chains that have been shed from the cell surface. The proteolysis of human syndecan-1 occurs at a number of specific sites (FIG. 25), by different proteases: the membrane-associated matrix metalloprotease MT1-MMP results in cleavage at Gly82-Leu83 and Gly245-Leu246; Thrombin cleaves at Arg126-Glu127 (ETTQL); Plasmin cleaves at Arg230-Asn231 (NQSPV); MMP2 cleaves at Gly82-Leu83 (LEATA), and MMP3 cleaves at bot Asp236-G1n237 (QGATG) and Gly245-Leu246 (LLDIP).

The Syndecan-1 antibody to the peptide EPKQANGGAYQKPTK (SEDQ ID NO. 6) is a unique and novel antibody, directed to the cytoplasmic region of Syndecan-1, allowing distinct detection of membrane associated Syndecan-1 protein allowing for the detection of advanced prostate cancer (see for example FIGS. 9, 10, 14-17).

### Example 4—The Biomarkers APPL1, Sortilin-1 and Syndecan-1 Can Also be Detected in Plasma

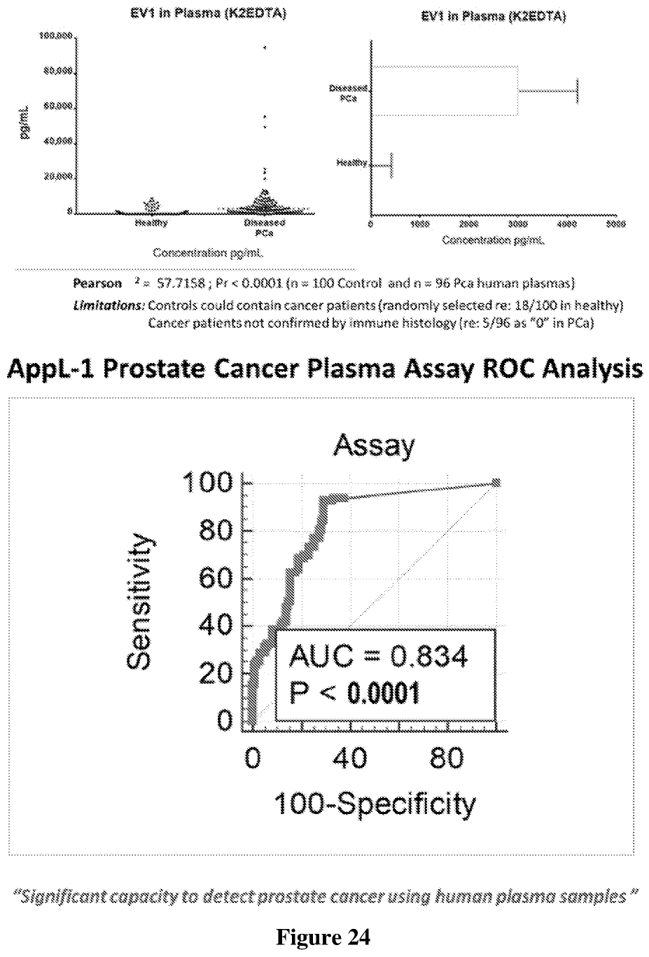

(i) APPL1, Sortilin-1 and Syndecan-1 provide a comprehensive set of biomarkers that detect key aspects of the pathogenesis in prostate cancer tissue, but can also be detected in blood (e.g. APPL1 in FIG. 24), giving for the first time a set of cancer biomarkers that directly report on the primary pathogenesis independent of a biopsy; no other prostate cancer biomarkers can achieve this important outcome. We have developed, validated and tested an APPL1 immunoassay that shows high sensitivity (≥95%) and specificity (≥93%) in blood plasma for the detection of prostate cancer (FIG. 24).

### (ii) Methods

The method for the determination of the Adaptor protein, phosphotyrosine interacting with PH domain and leucine zipper 1 (APPL-1) has a sandwich format. MSD standard bind plates are coated with an anti-APPL-1 antibody and then blocked to minimise non-specific binding. Human plasma samples are added, and the plate incubated. APPL-1 present in the plasma is bound by the immobilised coating antibody and unbound substances are washed away. APPL-1 is detected by a subsequent addition of a sulfo-tagged anti-APPL-1 (SEQ ID NO.2) antibody. Electrochemiluminescence signal is produced by addition of Read buffer and signal is measured on the MSD QuickPlex SQ 120. The signal produced is proportional to the amount of analyte present and interpolated from the calibration curve present on each plate.

(iii) Reagents:
Tween-20—Sigma Aldrich Cat. #P1379; Bovine Serum Albumin (BSA)—Sigma Aldrich Cat. #A7030; PBS—Sigma Aldrich Cat. #P5368, P3813, P38135; Ultra-pure water using Arium® pro UV/DI water purification system (Sartorius); MSD GOLD Read Buffer—Cat. #R92TG; Control Matrix: Human plasma K2EDTA individual and pooled stored at nominal −20° C. or −80° C.; Coating Buffer: PBS, 1× (phosphate buffered saline); Wash Buffer (WB): PBS/T (1×PBS with 0.05% Tween 20); Assay Buffer (AB): 1% BSA in PBST; MSD GOLD Read Buffer: Supplied ready to use.
Coating antibody Solution (APPL1 (SEQ ID NO.1) antibody, 2.00 μg/mL). Prepare coating antibody solution by diluting anti-APPL1 (SEQ ID NO.1) antibody in coating buffer as below.

| Stock ID/Number | Concentration | Spiking | Spiking Solution | Volume of Coating |
|---|---|---|---|---|
| of plates | (μg/mL) | Solution ID | Volume (□L) | Buffer (μL) |
| 1 | 2.00 | COAT | 12 | 5,898 |
| 2 | | COAT | 24 | 11,796 |
| 3 | | COAT | 36 | 17,694 |

Detection antibody Solution (APPL1 (SEQ ID NO.2) antibody-STag, 125 ng/mL). Prepare detection antibody solution by diluting anti-APPL1 (SEQ ID NO.2) antibody-STag in assay buffer as below. APPL1 (SEQ ID NO.2) antibody-STag is supplied at 400 μg/mL (DET).

| Stock ID/ Number | Concentration | Spiking | Spiking Solution | Volume of Assay |
|---|---|---|---|---|
| of plates | (ng/mL) | Solution ID | Volume (□L) | Buffer (μL) |
| DET_B | 4,000 | DET | 10 | 990 |
| 1 | 125 | DET_B | 185 | 5,735 |
| 2 | | DET_B | 375 | 11,625 |
| 3 | | DET_B | 750 | 23,250 |

Calibration Curve Samples: Prepare calibration curve by spiking APPL-1 protein into assay buffer on ice, as below.

| | | | Spiking | Volume of |
|---|---|---|---|---|
| | | Spiking | Solution | Assay |
| Stock ID/ | Concentration | Solution | Volume | Buffer |
| Standard ID | (pg/mL) | ID | (□L) | (μL) |
| STD_A | 6,400,000 | REF | 10 | 990 |
| BS01 (anchor) | 500,000 | STD_A | 50 | 590 |
| BS02 (ULOQ) | 300,000 | STD_A | 15 | 305 |
| BS03 | 250,000 | BS01 | 200 | 200 |
| BS04 | 50,000 | BS01 | 40 | 360 |
| BS05 | 10,000 | BS03 | 16 | 384 |
| BS06 | 2,000 | BS04 | 16 | 384 |
| BS07 | 400 | BS05 | 16 1 | 384 |
| BS08 | 100 | BS06 | 20 | 380 |
| BS09 (LLOQ) | 75.0 | BS06 | 15 | 385 |
| BS10 (anchor) | 50.0 | BS06 | 10 | 390 |
| Blank/NSB | — | — | — | 400 |

Quality/Positive Control Samples: Prepare buffer quality control samples by spiking APPL-1 protein into assay buffer on ice. Buffer quality control samples will be prepared fresh on the day of analysis.

| | | | Spiking | Volume of |
|---|---|---|---|---|
| | | Spiking | Solution | Assay |
| Stock ID/ | Concentration | Solution | Volume | Buffer |
| QC ID | (pg/mL) | ID | (□L) | (μL) |
| QC_A | 6,400,000 | REF | 10 | 990 |
| BQCULOQ | 300,000 | QC_A | 30 | 610 |
| BQCH | 225,000 | QC_A | 45 | 1235 |
| BQCM | 2,500 | BQCH | 10 | 890 |
| BQCL | 150 | BQCM | 45 | 705 |
| BQCLLOQ | 75.0 | BQCL | 250 | 250 |

Plasma Quality Control Samples: Prepare plasma quality control samples (PQC) by screening individual or pooled plasma and pooling all plasma with detectable level of APPL-1 to create anew pool with endogenous level of APPL-1. Plasma quality control will be diluted in assay buffer on ice.
Preparation of selectivity samples: Prepare selectivity samples by spiking APPL-1 protein into individual human plasma on ice as outlined in table below. Selectivity samples can be prepared in advance and stored frozen at nominal −80° C. in single use aliquots or prepared fresh on the day of analysis.

| | | | Spiking | Volume |
|---|---|---|---|---|
| | | | Solution | of |
| Stock ID/ | Concentration | Spiking | Volume | Diluent* |
| QCID ** | (pg/mL) | Solution ID | (μL) | (μL) |
| SEL_A | 6,400,000 | REF | 10 | 990 |
| SEL_B | 4,500,000 | SEL_A | 225 | 95 |
| SEL_C | 120,000 | SEL_B | 10 | 365 |
| SEL_D | 3,000 | SEL_C | 10 | 390 |
| SEL_E | 1,500 | SEL_C | 10 | 790 |
| SLPQCH | 225,000 | SEL_B | 10 | 190 |
| SLPQCL | 150 | SEL_D | 10 | 190 |
| SLPQC-LLOQ | 75.0 | SEL_E | 10 | 190 |
| SLPQC-BLK | — | Assay | 10 | 190 |
| | | Buffer | | |

Preparation of parallelism samples: Prepare parallelism samples by serially diluting ultrahigh human plasma on ice. Parallelism samples will be prepared fresh on the day of analysis.

| | | | Spiking | |
|---|---|---|---|---|
| | | Spiking | Solution | Volume of |
| | | Solution | Volume | Assay Buffer |
| ID | Total Dilution | ID | (μL) | (μL) |
| PAPQC-01 | 1 in 5 | Neat sample | 100 | 400 |
| PAPQC-02 | 1 in 10 | PAPQC-01 | 250 | 250 |
| PAPQC-03 | 1 in 20 | PAPQC-02 | 250 | 250 |
| PAPQC-04 | 1 in 40 | PAPQC-03 | 250 | 250 |
| PAPQC-05 | 1 in 80 | PAPQC-04 | 250 | 250 |

### (iv) Sample Preparation

Prior to analysis, all plasma samples will be subjected to recommended sample dilution 1 in 5 in assay buffer on ice as below.

| | | Spiking | |
|---|---|---|---|
| | Spiking | Solution | Volume of |
| | Solution | Volume | Assay Buffer |
| Dilution | ID | (μL) | (μL) |
| 1 in 5 | Neat plasma | 30 | 120 |

### (v) Assay Procedure

Plate Coating: Add 50 μL of coating solution to each well of the plate. Tap the side of the plate gently to distribute the solution across whole plate. Seal and incubate at nominal 4° C., for 14-24 hrs.
Plate Blocking: Remove the plate from the refrigerator (nominal 4° C.) and wash with 3×350 μL of wash buffer. Tap dry on paper towel. Add 150 μL of blocking buffer to each well of the plate. Seal and incubate at room temperature (no shaking) for minimum 60 minutes.
Sample preparation: Prepare samples (including calibration curve, QC and any validation samples as required) on ice. Dilute all plasma samples 1 in 5.
Sample Incubation: Wash with 3×350 μL of wash buffer. Tap dry on paper towel. Using multichannel pipette, add 50 μL of each sample in duplicate directly from tubes to the plate. Seal and incubate at room temperature for 90±10 minutes with shaking (450 rpm).
Detection: Prepare Detection Antibody Solution as per Section 2.5.7 of ALM. Wash plate with 3×350 μL of wash buffer. Tap dry on paper towel. Add 50 μL of detection solution to each well of the plate. Seal and incubate at room temperature for 60±10 minutes with shaking (450 rpm).
Plate reading: Wash with 3×350 μL of wash buffer using MSD wash program. Tap dry on paper towel. Add 150 μL of GOLD Read Buffer to each well of the MSD plate. Read on the MSD Plate Reader within 10 minutes of adding the ReadBuffer.

### Example 5—Determination of Syndecan-1 in Human Plasma by LC/MS/MS

### (i) Sample Preparation and Extraction Procedure:

Allow the plasma to thaw at nominal 4° C./ice slurry. Aliquot (in processing order) 25 μL blank samples, calibration curve samples, QC samples and subject samples into 1.5 mL microcentrifuge tubes. Vortex plasma samples vigorously to mix. Add 50 μL of Digest Buffer (DB, 50 mM NH4 Bicarbonate) to each of the wells. To any blank sample, add 10 uL PPDS. Add 10 μL of Working IS Solution (WIS, 10 ng/mL SIL-79) to each of the wells (except any blanks). Vortex briefly to mix e.g. 2*1 second with pause in between to allow liquid to settle at the bottom.
Place in ice slurry to equilibrate before enzyme digestion. Freshly prepare Trypsin Solution (TS) at 10.0 mg/mL just prior to first addition. Add 1.0 mL (Note: Adjust volume of TD (Typsin Diluent) to exactly match actual weight of Trypsin to give 10.0 mg/mL) of cold TD to a pre-weighed frozen ˜10 mg Trypsin in a 5mL Protein Low-bind Eppendorf vial. Vortex briefly to dissolve. Store in ice slurry. Note: Trypsin Diluent ˜1 mm HCL, 20 mM CaCl2 in MilliQ water. Add 10.00 μL of freshly prepared TS to the prepared sample above. Conduct all the following steps for each Sample before proceeding with the next sample.
Conduct addition of TS in batches of 20 i.e. >=10 minutes after completing addition of TS to the first sample of the batch, start the addition of the THS (Trypsin Halting Solution). Vortex 2×, with a pause between to allow liquid to drain to the bottom. Add 10.00 μL of THS to the prepared sample. Vortex 2×, with a pause between to allow liquid to drain to the bottom.
Using a Multipette, add 1.0 mL of LiCl-4 deg to each Eppendorf vial, vortex 2× and place in −80 Freezer for at least 30 minutes.
Flushing: Remove required SPE solutions from cold room storage to allow to come to room temperature. Remove diluted digested plasma from −80 storage and place in ice slurry to thaw. Start minicentrifuge to equilibrate to 4 degrees ready for spinning. CBA—Agilent Bond Elute CBA cartridge, 100 mg, 1 mL, 40 um. Format: Straight barrel cartridge. Part #12102011. Allow all solution additions to the SPE cartridges to flow by gravity alone. Very slightly push using a 50 mL syringe if the flow has obviously halted and record the observation. Using a Multipette, add 1.0 mL of TB (Tris Buffer, 0.1M Tris Buffer, pH 8.7) to each CBA cartridge. Then, add 1.0 mL of F1 (Flush #1, 30% Acetonitrile/1% TFA) to each CBA cartridge. Add 1.0 mL of F2 (Flush #2, 80% Acetonitrile/1% TFA) to each CBA cartridge.
Conditioning: Using a Multipette, add 1.0 mL of Methanol to each CBA cartridge. Using a Multipette, add 1.0 mL of Methanol to CBA cartridges in groups of 3 and push strongly to expel any residual air. Ensure the CBA sorbent bed does not drain of ANY liquid i.e. at all times keep the liquid level above the sorbent bed top frit.
Equilibration: Using a Multipette, add 1.0 mL of LiCl-RT (100 mM LiCl to be used at room temperature (to each CBA cartridge. Using a Multipette, add 1.0 mL of LiCl-RT (100 mM LiCl to be used at room temperature to each CBA cartridge).
Sample Load: Vortex diluted digested plasma, pause, repeat, ensuring air vortex goes to the bottom for complete mixing. Spin for 10 minutes at 13.2 k and 4 degrees. Place microcentrifuge tube with the hinge facing outward. Place back in ice slurry. Proceed with Sample loading without undue delay. Transfer CBA cartridges from the SPE manifold/s to individually labelled 15 mL blue top falcon tubes. Remove 1.0 mL Supernatant. Angle the microcentrifuge tube at 45 degrees with the hinge upward.

Carefully remove the supernatant from the top of the liquid furthest from the bottom to ensure no ppt is sampled (may block SPE cartridge).
Washing: Add 1.0 mL of LiCl-RT (100 mm LiCl to be used at room temperature (to each CBA cartridge. Add 1.0 mL of 10-Tris (10% TB/90% 100 mM LiCl) to each CBA cartridge.
Discard: Using a Multipette, add 200 uL of 50-Tris (50% TB/50% 100 mM LiCl) to each CBA cartridge.
Elution: Add 10 uL Keeper (Keeper—undiluted plasma precipitate solution) to the bottom of 10 mL tubes used to capture the elution step. Transfer CBA cartridges from the 15 mL blue top falcon pp tubes to 10 mL tube. Using a Multipette, add 250 uL of 50-Tris to each CBA cartridge. Using a Multipette, add 250 uL of 50-Tris to each CBA cartridge. Remove SPE Cartridges and seal in 50 mL blue falcon tubes and stored at 4 degrees. Ensure any hanging drops are shaking off BEFORE removal of the SPE cartridge from the 10 mL yellow top elution tube. Vortex elution tubes 3× with distinct pause in between, to allow all liquid to drain to the bottom each time. Transfer 500 uL of eluent to 0.5 mL tapered pp HPLC vial and cap. Inspect the tapered bottom of each vial and ensure there is not any air bubbles. If so, tap the bottom of the vial with your finger until the air bubbles are released.

### Example 6—Diagnosis of Prostate Cancer Based on Changes in mRNA Expression

A diagnosis of the presence of prostate cancer may be made upon the basis of one or more of the level of mRNA expression of one or more of the mRNAs for any of the markers as described herein, the level of the marker proteins as described herein, the secretion of the marker proteins as described herein, the presence of the marker proteins in a biological fluid as described herein, or on the basis of immunohistology on tissue or biopsy samples of any of the marker proteins as described herein.

Examples of selected markers that may be used include one or more of the following proteins or their mRNAs: CATHEPSIN B, CAPTHESIN D, α-GALACTOSIDASE, RAB7, LIMP-1, LIMP-2, TFR1, TFR2, STAMP2, SORT1 (SORTILIN), APPL1, EEA-1, LAMP-1, RAB4, APPL2, RABS, RAB11, RAB21, Myosin VI, OCRL, GIPC1, MPR, PAP, ACTIN, GLUT1, GLUT4, LPL, OSBP, PGRN/GRN, NTS, RAP, M6PR, IGFR2, MYO1B, PDCD6IP, SDCBP, SDC1, Survivin, ITGB3, ITGB5, STX7, STX12, EGFR, PDGF, VEGF's, FN1, VTN, PAI-1, laminins, BMP's, FGF1, FGF2, FGF3, FGFR1, FGFR2, FGFR3, NOX2, and NOX4.

For example, a cylindrical sample (biopsy) of prostate tissue may be removed through the rectum, using hollow needles, and a portion of the sample prepared for histology and immunohistochemistry. If the prostate is surgically removed, a pathologist may prepare a slice of the prostate tissue for analysis.

APPL1 may be selected as a suitable marker and analysis conducted as described in Example 1 using immunohistochemistry to determine the distribution of APPL1 using an APPL1 specific antibody. APPL1 maps the cancer delineates the cancer margins and shows dramatically increased staining within the tumour mass as the cancer progresses in grade grouping. Such staining would be indicative of the presence of prostate cancer and with high sensitivity and specificity be suitable for confirming diagnosis in clinical practice.

On the basis of the detection using a selected marker as described herein, a variety of treatment options are available, dependent upon the diagnosis and/or prognosis and the extent of recurrence of the cancer, in addition to, or in conjunction with, the prognostic value of the selected markers described herein:

### (i) Low Risk of Recurrence:

Treatment for patients with clinical stage T1-T2a, Gleason score 2-6, PSA <10 ng/mL, with a life expectancy <10 y, includes active surveillance

Treatment for patients with a life expectancy ≥10 y includes active surveillance, or radical prostatectomy (RP) with or without pelvic lymph node dissection (PLND) if predicted probability of lymph node metastases ≥2%; RP being a standard therapy for localized prostate cancer, involving the removal of the prostate and seminal vesicles with or without pelvic lymph nodes; this may be done using either open or laparoscopic (robotic-assisted) technique;

or

Radiation therapy for patients with localized disease, and 3-dimensional (3D) techniques such as 3D conformal radiation treatment (3D-CRT), which offer benefits such as reduced toxicity and the use of higher doses; second-generation techniques, including intensity-modulated radiation therapy (IMRT), may also be required, especially if doses ≥78 Gy are administered.

Radiation therapy doses of 75.6-79 Gy in conventional 36-41 fractions to the prostate with 3D-CRT/IMRT with daily image-guided radiotherapy (IGRT) or brachytherapy (recommended dose rate: 145 Gy for iodine-125 and 125 Gy for palladium-103).

Patients with low-risk cancer are typically not candidates for pelvic lymph node irradiation or androgen deprivation therapy (ADT).

### (ii) Intermediate Risk of Recurrence:

Treatment for patients with clinical stage T2b-T2c, Gleason score 7, PSA 10-20 ng/mL, who have a life expectancy <10 y, include active surveillance; or

Radiation therapy (doses of 78-80+ Gy) with 3D-CRT/IMRT with daily IGRT with or without short-term neoadjuvant/concomitant/adjuvant ADT for 4-6 months with or without brachytherapy (recommended dose rate: 145 Gy for iodine-125 and 125 Gy for palladium-103).

Treatment recommendations for patients with a life expectancy ≥10 y includes RP with PLND if predicted probability of lymph node metastasis ≥2% or radiation therapy (doses of 78-80+ Gy) with 3D-CRT/IMRT with daily IGRT with or without short-term neoadjuvant/concomitant/adjuvant ADT for 4-6 months with or without brachytherapy (recommended dose rate: 145 Gy for iodine-125 and 125 Gy for palladium-103).

Intermediate-risk cancers consider combining brachytherapy (recommended dose rate: 145 Gy for iodine-125 and 125 Gy for palladium-103) with EBRT (40-50 Gy) with or without 4-6 mo neoadjuvant/concomitant/adjuvant ADT.

Administering ADT before, during, and after radiation prolongs survival in patients.

(iii) High Risk of Recurrence:
Clinical Stage T3a, Gleason Score 8-10, PSA >20 ng/mL

Treatment options include radiation therapy (doses of 78-80+ Gy) with 3D-CRT/IMRT plus long-term neoadjuvant/concomitant/adjuvant ADT for 2-3 y, or radiation therapy (doses of 78-80+ Gy) with 3D-CRT/IMRT with daily IGRT plus brachytherapy (recommended dose rate: 145 Gy for iodine-125 and 125 Gy for palladium-103) with or without short-term neoadjuvant/concomitant/adjuvant ADT for 4-6 months, or RP plus PLND for selected patients with no fixation.

High-risk cancers may be treated with combination EBRT (40-50 Gy) and brachytherapy with or without 4-6 months neoadjuvant/concomitant/adjuvant ADT.

### Example 7—Envision Sciences Monoclonal Antibodies to Syndecan-1 are Specific Biomarkers for the Detection of Prostate Cancer Pathogenesis

Anti-Syndecan-1 mouse monoclonal antibodies were generated (Genscript, Piscataway, NJ 08854, USA.) using the peptide sequence EPKQANGGAYQKPTK (SEQ ID NO 6), SHPHRDMQPGHHETS (SEQ ID NO: 7), and TPRPRETTQLPT (SEQ ID NO: 8). These antibodies were compare to the commercially available antibodies ab34164 (abcam; mouse monoclonal [B-A38] to Syndecan-1; www.abcam.com/syndecan-1-antibodyb-a38-ab34164.html) and ab128936 (abcam; recombinant anti-Syndecan-1 antibody [EPR6454]; https://www.abcam.com/syndecan-l-antibody-epr6454-ab128936.htm).

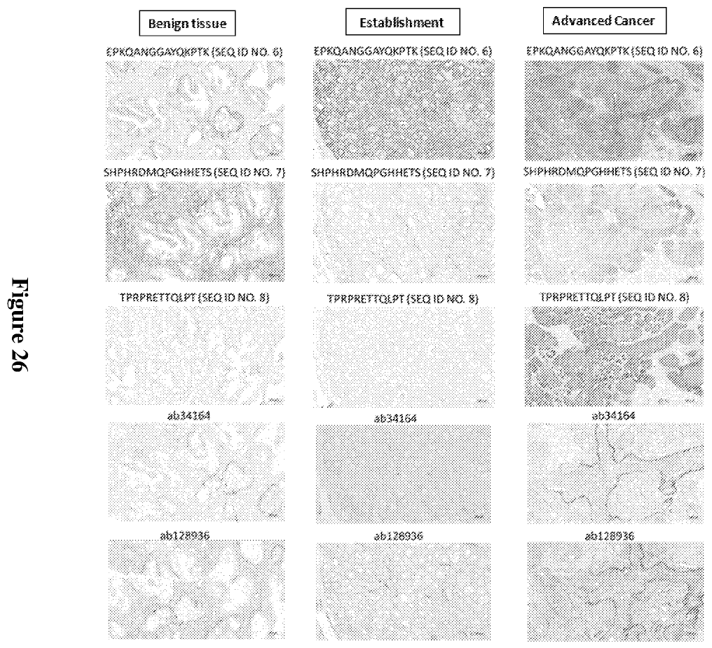

A comparison of immunohistochemistry performed with different commercially available antibodies (ab34164 and ab128936) and Envision Sciences monoclonal antibodies (SEQ ID's 6, 7 and 8) demonstrated that only two of the Envision Sciences monoclonal antibodies could accurately depict prostate cancer pathogenesis in patient tissue samples. The commercial antibodies to Syndecan-1 and the Envision Sciences SEQ ID NO: 7 were not able to define the pathogenesis in prostate cancer compared to benign tissue (FIG. 26). In contrast two of the Envision Sciences monoclonal antibodies (SEQ ID NOs: 6 and 8) accurately detected advanced prostate cancer and could distinguish benign and cancer tissue (FIG. 2). Thus despite the antibodies being directed against the same target protein only two specific linear sequences on Syndecan-1 were able to depict the pathogenesis in prostate cancer patient tissue samples.

On the basis of the advanced cancer detection using the Envision Sciences monoclonal antibodies described herein, a variety of treatment options are available, dependent upon the diagnosis and/or prognosis and the extent of recurrence of the cancer, in addition to, or in conjunction with, the prognostic value of the selected markers described herein.

In reference to prior art obtained with other Syndecan-1 antibodies it is apparent that only the specific linear sequences detected by the two Envision Sciences monoclonal antibodies is able to accurately depict prostate cancer pathogenesis. Although the present disclosure has been described with reference to particular examples, it will be appreciated by those skilled in the art that the disclosure may be embodied in many other forms.

### Example 8—The Biomarkers APPL1, Sortillin-1 and Syndecan-1 Equally as Well in Both Needle Core Biopsy Sections and Prostatectomy Sections

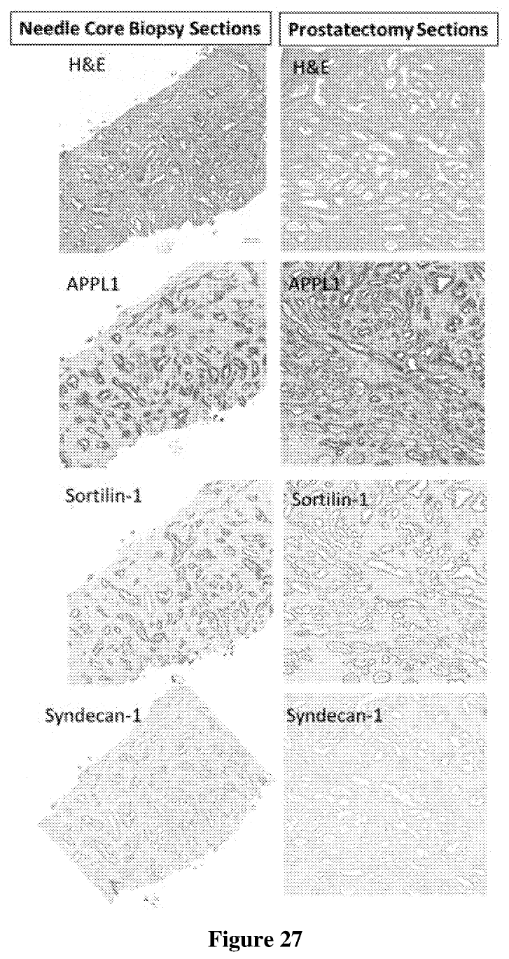

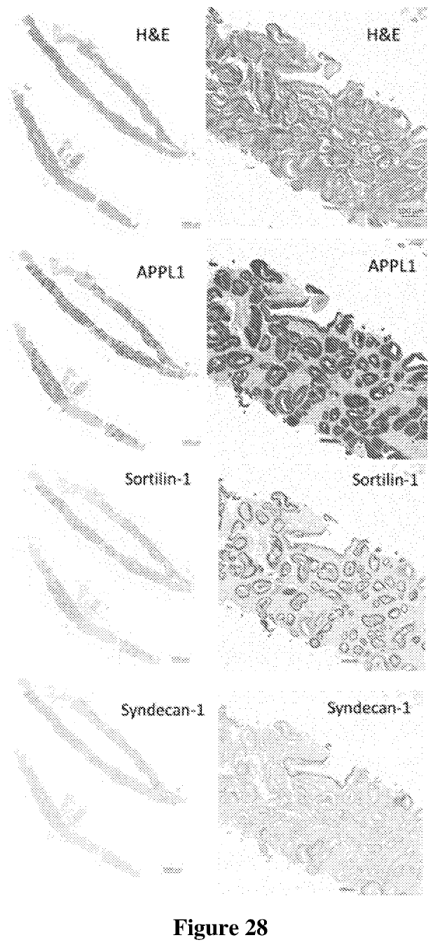

APPL1, Sortilin-1 and Syndecan-1 provide a comprehensive set of biomarkers that detect key aspects of the pathogenesis equally well in different prostate cancer tissue samples including needle biopsies and prostatectomies. FIG. 27 shows that APPL1, Sortilin-1 and Syndecan-1 have similar capacity to depict the pathogenesis in needle biopsies and prostatectomy samples. The pathology depicted in FIG. 27 shows IHC detection with APPL1 and Sortilin-1 but not Syndecan-1 and is representative of a patient that would be recommended for active surveillance. A second example of a patient that is suitable for active surveillance is depicted in FIG. 28 and again shows APPL1 and Sortilin-1 but not Syndecan-1. The biomarkers Sortilin-1 and Syndecan-1 can clearly identify patients with ISUP grade group 1 (establishment cancer) that are suitable for active surveillance (FIG. 27, 28). Although the present disclosure has been described with reference to particular examples, it will be appreciated by those skilled in the art that the disclosure has direct application for prostate cancer pathology detection in different patient prostate tissue samples.

### Example 9—Use of Sortilin-1 Antibodies to WVSKNFGGKWEEIHK (SEQ ID NO: 4) and EKDYTIWLAHSTDPE (SEQ ID NO: 5) Demonstrating That Only One Specific Epitope Detects Establishment Prostate Cancer Pathogenesis

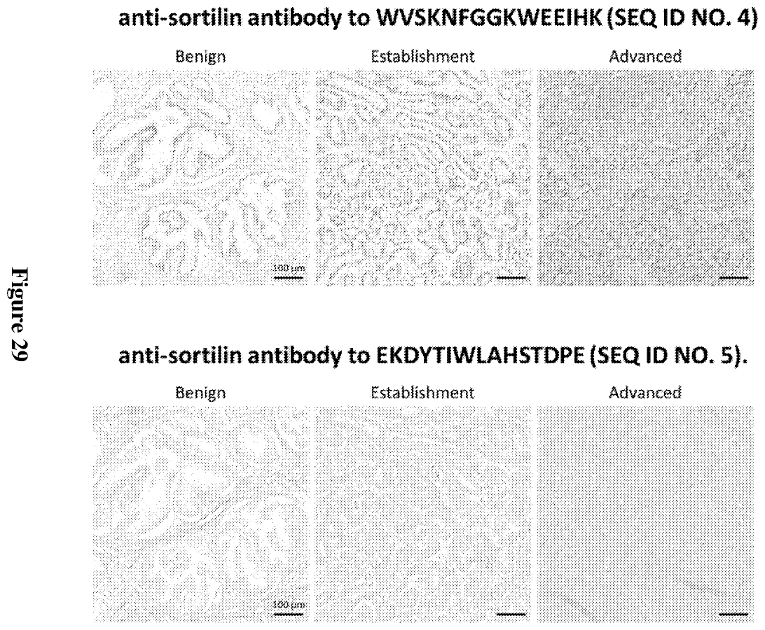

(i) Use of Sortilin-1 peptide WVSKNFGGKWEEIHK (SEQ ID NO: 4) is the optimum peptide for antibody production to Sortilin-1 on the basis of the optimal detection of establishment cancer. The Sortilin-1 antibody to the peptide WVSKNFGGKWEEIHK (SEQ ID NO: 4) is a unique and novel antibody, directed to the extracellular region of Sortilin-1, allowing distinct detection of Sortilin-1 protein in establishment prostate cancer (FIG. 29). This antibody has minimal labelling in benign secretory cells (punctate and supranuclear) and it does not label basal cells (FIG. 29). In establishment prostate cancer, Sortilin-1 antibody is abundant in secretory cells (punctate and supranuclear). In advancer cancer, Sortilin-1 labelling is granular and no longer supranuclear in its position, with some cytoplasmic distribution.
(ii) Sortilin-1 peptide; EKDYTIWLAHSTDPE (SEQ ID NO: 5) is another peptide used for antibody production to Sortilin-1. The anti-Sortilin-1 monoclonal antibody to the peptide EKDYTIWLAHSTDPE (SEQ ID NO: 5) is also directed to the extracellular region of Sortilin-1, but does not effectively recognize either establishment or advanced prostate cancer (FIG. 29). This antibody does not label secretory and basal cells in benign glands; and while in establishment and advanced prostate cancer, this antibody has cytoplasmic distribution it is not optimal for the detection of prostate cancer pathogenesis. (FIG. 29).

The Sortilin-1 peptide; WVSKNFGGKWEEIHK (SEQ ID NO: 4) is the optimum peptide for antibody production to Sortilin-1 on the basis of the optimal detection of establishment cancer, which is not evident in any prior art investigating Sortilin-1 in prostate cancer patient tissues.

Reference to any prior art in this specification is not, and should not be taken as, an acknowledgment or any form of suggestion that this prior art forms part of the common general knowledge in any country.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used herein, the singular forms “a”, “an” and “the” include plural aspects unless the context already dictates otherwise.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

Although the present disclosure has been described with reference to particular examples, it will be appreciated by those skilled in the art that the disclosure may be embodied in many other forms.

1. A method of detecting a prostate cancer in a subject, the method comprising detecting one or more of an altered presence, level, secretion and distribution of at least the following three endosomal biomarkers APPL1 (Adaptor protein, phosphotyrosine interacting with PH domain and leucine zipper 1, APPL1), Sortilin-1 (SORT1), and Syndecan-1 (SDC1) in a biological sample from the subject as compared to a reference.

2. The method of claim 1, wherein the reference is a normal or benign prostate tissue or a normal blood or plasma sample.

3. The method of claim 1, wherein the method comprises detecting elevated levels of APPL1, SORT1 and SDC1.

4. The method of claim 1, wherein the endosomal biomarkers are proteins or nucleic acids.

5. A method of detecting and measuring the severity of a prostate cancer in a subject, the method comprising detecting one or more of an altered presence, level, secretion and distribution of at least the following three endosomal biomarkers: APPL1, SORT1, and SDC1, from a biological sample of the subject as compared to a reference.

6. The method of claim 5, wherein the method comprises determining the progression of the prostate cancer in the subject.

7. A method of monitoring the progression of a prostate cancer in a subject, the method comprising detecting one or more of an altered presence, level, secretion and distribution of at least the following three endosomal biomarkers: APPL1, SORT1, and SDC1 in a biological sample from the subject as compared to a reference; or A method of detecting a prostate cancer in a subject, the method comprising:
obtaining a biological sample from the subject;
processing the biological sample to allow detection of at least the following three endosomal markers: APPL1, SORT1, and SDC1;
detecting, in each biomarker, one or more of an altered presence, level, secretion and distribution of the selected biomarker in the processed sample; and
identifying a prostate cancer and/or the progression of the cancer in the subject; or a method of detecting a prostate cancer in a subject, the method comprising:
obtaining a biological sample from the subject;
processing the biological sample to allow detection of at least the following three endosomal markers: APPL1, SORT1, and SDC1;
detecting, in each biomarker, one or more of an altered presence, level, secretion and distribution of the selected biomarker in the processed sample; and
identifying a prostate cancer and/or the progression of the cancer in the subject; or a method for detecting a prostate cancer in a subject, the method comprising:
obtaining a biological sample from the subject;
processing the biological sample to allow detection of at least the following three endosomal markers: APPL1, SORT1, and SDC1;
comparing, in each biomarker, the presence, level, secretion and distribution of the selected markers; optionally with one or more other markers known to be indicative of the presence or absence of prostate cancer in the subject; and
identifying prostate cancer in the subject; or
a method of detecting a prostate cancer in a subject, the method comprising:
processing a biological sample from said subject to allow detection of at least the following three endosomal markers: APPL1, SORT1, and SDC1;
comparing the presence, level, secretion and distribution of the selected markers;
optionally with one or more other markers known to be indicative of the presence or absence of prostate cancer in the subject; and
identifying prostate cancer and/or the progression of the cancer in the subject; or a method of detecting a prostate cancer in a subject, the method comprising:
obtaining a biological sample from the subject;
processing the sample to allow detection of at least the following three endosomal markers: APPL1, SORT1, and SDC1; and
detecting one or more of an altered presence, level, secretion and distribution of these selected markers in the processed sample; and
identifying a prostate cancer and/or the progression of the cancer in the subject, wherein the subject is effectively treated for the prostate cancer according to the biomarker pattern; or a method for diagnosing and treating a prostate cancer in a subject, the method comprising:
detecting APPL1, SORT1, and SDC1 from the subject; and
treating the subject based on one or more of the presence, level, secretion and distribution of the selected markers detected, with a cancer therapy; or a method of determining the likelihood of the presence of a prostate cancer in a subject, the method comprising detecting one or more of an altered presence, level, secretion and distribution of at least the following three endosomal biomarkers APPL1, SORT1, and SDC1 in a biological sample from the subject as compared to a reference; or a method of identifying a subject suffering from prostate cancer who is likely to be responsive to a cancer therapy, the method comprising detecting one or more of an altered presence, level, secretion and distribution of at least the following three endosomal biomarkers APPL1, SORT1, and SDC1 in a biological sample of the subject as compared a reference; or a method of predicting the risk of recurrence of prostate cancer in a subject following a cancer therapy, the method comprising detecting one or more of an altered presence, level, secretion and distribution of at least the following three endosomal biomarkers APPL1, SORT1, and SDC1 in a biological sample of the subject as compared a reference.

8-15. (canceled)
